(19) United States
(12) Patent Application Publication
(10) Pub. No.: US 2025/0037825 A1
(43) Pub. Date: Jan. 30, 2025

(54) DEEP LEARNING PLATFORM AND APPLICATION FOR CATARACT AND REFRACTIVE SURGERY GUIDANCE

(71) Applicant: Oculotix, Inc., Menlo Park, CA (US)

(72) Inventor: KRISHNAKUMAR VENKATESWARAN, Aliso Viejo, CA (US)

(73) Assignee: Oculotix, Inc., Menlo Park, CA (US)

(21) Appl. No.: 18/783,759

(22) Filed: Jul. 25, 2024

Related U.S. Application Data
(60) Provisional application No. 63/515,520, filed on Jul. 25, 2023.
(60) Provisional application No. 63/627,301, filed on Jan. 31, 2024.

(51) Int. Cl. G16H 20/00; G16H 10/60

(52) U.S. Cl. CPC G16H 20/00; G16H 10/60

(57) ABSTRACT

A method and system for managing treatment of an ophthalmic patient is provided. In one method, optical information is collected from a patient. A deep learning model hosted on an ophthalmic treatment platform is trained using training data including historical ophthalmic procedure data associated with a plurality of patients, complication data associated with the plurality of patients, and patient survey data regarding treatment satisfaction of the plurality of patients. The platform is configured to generate recommendations and/or predictions at varying stages of treatment. At least at a first stage of treatment, the platform is configured to generate a treatment recommendation regarding an ophthalmological treatment, the treatment recommendation including an ophthalmic lens type recommendation and a probability of a predetermined surgical outcome associated with the ophthalmic lens type recommendation.

### CROSS-REFERENCE TO RELATED APPLICATIONS

The present application claims priority from U.S. Provisional Patent Application No. 63/627,301, filed on Jan. 31, 2024, and U.S. Provisional Patent Application No. 63/515,520, filed on Jul. 25, 2023, the disclosures of each of which are hereby incorporated by reference in their entireties.

### BACKGROUND

Prior to cataract surgery, an optical technician (e.g., an ophthalmologist) must select a synthetic lens to be implanted, which will replace the clouded natural lens of the human eye. This selection process is typically based on the prospective patient's preoperative measurements and the ophthalmologist's experience from previous procedures performed on other patients. Consequently, ophthalmologists may be reluctant to recommend new or more expensive advanced lenses due to the multitude of available options and the uncertainty of patient outcomes with respect to those options. Along with precise intraocular lens (IOL) power calculations, other clinical assessments such as patient's biometry, ocular and general health and patient's lifestyle drive post operative visual satisfaction of the patient. Although modem formulas have refined the previous calculation methods, accurate formulas by itself do not guarantee patient satisfaction. patient's postoperative visual satisfaction heavily rely on the accuracy of biometry devices, various preoperative subjective clinical assessments, patient's lifestyle along with errors introduced during manufacturing process of the lenses.

Still further, and generally speaking, an optical element such as human cornea includes lower order optical aberrations, such as spherical and cylindrical powers, as well as higher order aberrations such as coma, spherical aberration. IOL manufacturers manufacture lenses over a spherical power range, typically between 0 Diopters to 35 Diopters, in steps of 0.5 Diopters and cylinder power range of 0 Diopters to 6 Diopters in steps of 0.5 Diopters. A spherical aberration on the manufactured IOL is a constant value across the entire power range. For instance, some IOL manufacturers maintain a spherical aberration value of −0.1 micron or −0.2 micron across their entire range of stock keeping units (SKU) manufactured at their facility. Apart from the quantization of key parameters such as sphere, cylinder and spherical aberrations, other higher order aberrations, pupil size dependency of lens performance are all neglected in standard intraocular lens designs. Therefore, intraocular lenses are designed within a specific range of spherical, cylindrical powers to fit the entire human population. Spherical aberration in such lens designs is treated similarly.

### SUMMARY

In general terms, the present disclosure relates to an artificial intelligence (AI) and deep learning (DL) platform that is capable of receiving parameters from a plurality of previous patients and procedures, and providing recommendations regarding optical lenses, techniques, and information regarding likely outcomes to ophthalmologists and patients. Such a system is capable of weighing a number of parameters based on past patient and procedure information, and can provide a lens recommendation based on particular inputs of a prospective patient. The DL/AI platform may include a deep-learning engine with expandable neural net capable of evaluating which pre-operative factors will contribute to the vision outcome of the patient. In some example aspects, a custom lens design may be generated using such artificial intelligence and deep learning techniques.

In one example aspect, a computer-implemented method of managing treatment of an ophthalmic patient is provided. The method includes collecting optical information from a patient wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients. The method further includes generating, at a first stage of treatment, a treatment recommendation regarding an ophthalmological treatment, the treatment recommendation including an ophthalmic lens type recommendation and a probability of a predetermined surgical outcome associated with the ophthalmic lens type recommendation.

In a second aspect, an ophthalmic treatment recommendation and guidance platform implemented on a computing system is provided the computing system includes a processor and a memory communicatively coupled to the processor. The memory storing instructions that, when executed by the processor, cause the platform to: collect optical information from a patient wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients; and generate, at a first stage of treatment, a treatment recommendation regarding an ophthalmological treatment, the treatment recommendation including an ophthalmic lens type recommendation and a probability of a predetermined surgical outcome associated with the ophthalmic lens type recommendation.

In a further aspect, a computer-implemented method of managing treatment of an ophthalmic patient includes collecting optical information from a patient, wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients. The method further includes generating, at the deep-learning model, a treatment recommendation regarding an ophthalmological treatment, the treatment recommendation including a custom intraocular lens design recommendation.

### BRIEF DESCRIPTION OF THE DRAWINGS

FIG. 1 illustrates an example environment in which aspects of the present disclosure may be implemented.

FIG. 2 illustrates an example computing system that may be used to implement aspects of the present disclosure.

FIG. 3 illustrates a training process for an AI model hosted by an ophthalmic treatment recommendation and guidance platform and useable to generate recommendations for treatment in accordance with example aspects of the present disclosure.

FIG. 4 illustrates a usage scenario for the AI model of FIG. 3.

FIG. 5 illustrates a logical diagram of an application that uses the AI model of FIGS. 3-4 to generate treatment recommendations and predictions, according to example aspects of the present disclosure.

FIG. 6 illustrates an example method of use of the ophthalmic treatment recommendation and guidance platform described herein.

FIG. 7 illustrates a patient data user interface presented on a caregiver computing system based on interaction with an application hosted by the ophthalmic treatment recommendation and guidance platform, according to an example embodiment.

FIG. 8 illustrates a further patient data user interface presented on a caregiver computing system based on interaction with an application hosted by the ophthalmic treatment recommendation and guidance platform, according to an example embodiment.

FIG. 9 illustrates a biometry user interface presented on a caregiver computing system based on interaction with an application hosted by the ophthalmic treatment recommendation and guidance platform, according to an example embodiment.

FIG. 10 illustrates a results user interface presented on a caregiver computing system based on interaction with an application hosted by the ophthalmic treatment recommendation and guidance platform, according to an example embodiment.

FIG. 11 illustrates a guidance user interface presented on a caregiver computing system based on interaction with an application hosted by the ophthalmic treatment recommendation and guidance platform, according to an example embodiment.

FIG. 12 illustrates a further guidance user interface presented on a caregiver computing system based on interaction with an application hosted by the ophthalmic treatment recommendation and guidance platform, according to an example embodiment.

FIG. 13 illustrates a further guidance user interface presented on a caregiver computing system based on interaction with an application hosted by the ophthalmic treatment recommendation and guidance platform, according to an example embodiment.

FIG. 14 illustrates a patient data user interface presented on a caregiver computing system based on interaction with an application hosted by the ophthalmic treatment recommendation and guidance platform, according to a further example embodiment.

FIG. 15 illustrates a clinical assessment user interface presented on the caregiver computing system based on interaction with an application hosted by the ophthalmic treatment recommendation and guidance platform, according to the further example embodiment.

FIG. 16 illustrates a preoperative clinical measurement user interface presented on the caregiver computing system based on interaction with an application hosted by the ophthalmic treatment recommendation and guidance platform, according to the further example embodiment.

FIG. 17 illustrates a questionnaire user interface presented on the caregiver computing system based on interaction with an application hosted by the ophthalmic treatment recommendation and guidance platform, according to the further example embodiment.

FIG. 18 illustrates a patient data gathering status user interface presented on the caregiver computing system based on interaction with an application hosted by the ophthalmic treatment recommendation and guidance platform, according to the further example embodiment.

FIG. 19 illustrates a surgery preparation user interface presented on the caregiver computing system based on interaction with an application hosted by the ophthalmic treatment recommendation and guidance platform, according to the further example embodiment.

FIG. 20 illustrates a further surgery preparation user interface presented on the caregiver computing system based on interaction with an application hosted by the ophthalmic treatment recommendation and guidance platform, according to the further example embodiment.

FIG. 21 illustrates a further surgery preparation user interface presented on the caregiver computing system based on interaction with an application hosted by the ophthalmic treatment recommendation and guidance platform, according to the further example embodiment.

FIG. 22 illustrates a guidance user interface presenting surgery risks presented on the caregiver computing system based on interaction with an application hosted by the ophthalmic treatment recommendation and guidance platform, according to the further example embodiment.

FIG. 23 illustrates a further guidance user interface presenting outcome predictions on the caregiver computing system based on interaction with an application hosted by the ophthalmic treatment recommendation and guidance platform, according to the further example embodiment.

FIG. 24 illustrates a further guidance user interface presenting further surgical outcome predictions on the caregiver computing system based on interaction with an application hosted by the ophthalmic treatment recommendation and guidance platform, according to the further example embodiment.

FIG. 25 illustrates a prediction user interface presented on the caregiver computing system based on interaction with an application hosted by the ophthalmic treatment recommendation and guidance platform, according to the further example embodiment.

FIG. 26 illustrates a surgical guidance user interface presented on the caregiver computing system based on interaction with an application hosted by the ophthalmic treatment recommendation and guidance platform, according to the further example embodiment.

FIG. 27 illustrates a training process for an AI model hosted by an ophthalmic treatment recommendation and guidance platform and useable to generate patient-specific customized lens designs for treatment in accordance with example aspects of the present disclosure.

FIG. 28 is a flowchart of an example method of generating a customized lens design using AI assistance within an ophthalmic treatment recommendation and guidance platform as described herein.

FIG. 29 illustrates a pre-operative manifest assessment user interface presented on the caregiver computing system based on interaction with an application hosted by the ophthalmic treatment recommendation and guidance platform, according to a further example embodiment.

FIG. 30 illustrates a pre-operative visual acuity assessment user interface presented on the caregiver computing system based on interaction with an application hosted by the ophthalmic treatment recommendation and guidance platform, according to a further example embodiment.

FIG. 31 illustrates a pre-operative clinical measurement user interface presented on the caregiver computing system based on interaction with an application hosted by the ophthalmic treatment recommendation and guidance platform, according to a further example embodiment.

FIG. 32 illustrates a further pre-operative clinical measurement user interface presented on the caregiver computing system based on interaction with an application hosted by the ophthalmic treatment recommendation and guidance platform, according to a further example embodiment.

FIG. 33 illustrates a further pre-operative clinical measurement user interface presented on the caregiver computing system based on interaction with an application hosted by the ophthalmic treatment recommendation and guidance platform, according to a further example embodiment.

FIG. 34 illustrates a further pre-operative clinical measurement user interface presented on the caregiver computing system based on interaction with an application hosted by the ophthalmic treatment recommendation and guidance platform, according to a further example embodiment.

FIG. 35 illustrates a recommendation user interface presented on the caregiver computing system based on interaction with an AI-assisted lens matching and/or design application hosted by the ophthalmic treatment recommendation and guidance platform, according to a further example embodiment.

FIG. 36 illustrates a detail user interface presented on the caregiver computing system in association with the recommendation illustrated in FIG. 35, according to that example embodiment.

FIG. 37 illustrates a prediction user interface presented on the caregiver computing system in association with the recommendation illustrated in FIG. 35, according to that example embodiment.

### DETAILED DESCRIPTION

In example implementations, an artificial intelligence (AI) platform is provided that is capable of receiving parameters from a plurality of previous patients and procedures, and providing recommendations regarding optical lenses, techniques, and information regarding likely outcomes to ophthalmologists and patients. Such a system is capable of weighing a number of parameters based on past patient and procedure information, and can provide a lens recommendation based on particular inputs of a prospective patient. The AI platform may include a deep-learning engine capable of evaluating which pre-operative factors will contribute to the vision outcome of the patient. Based on the extent that each factor will impact the outcome, the AI models provided by the AI platform can be trained to utilize relevant pre-operative information from a prospective patient in order to make a lens recommendation to the ophthalmologist. An optical mapping algorithm is also used to map a model of the selected lens onto the patient's eye to assist in determining the potential patient benefits associated with that lens selection, and patient vision outcome predictions accompany the lens recommendation. Subsequently, post-operative measurements may be input into the engine as a means of training the engine to produce improved recommendations for future patients.

In examples, patient progress may be tracked throughout a pre-operative process and post-operative treatments and/or outcomes to provide updated guidance based on a current state of the patient.

To place the present disclosure in context, numerous commercial biometry instruments are available for assessing the geometry and axial positioning of the eye's optical surfaces. When it comes to power calculations, advancements in biometry methods for determining eye geometry, such as the SRK formula, have been developed based on measurements of corneal power and axial length. Empirical constants, like the A-constant, were introduced because some of the eye's optical parameters, including the intraocular lens position, were not precisely known. Calculators such as RBF-Hill use advanced mathematical techniques to interpolate data and optimized to use for data within certain bounds.

Modem formulas have refined the older calculation methods to account for uncertainties resulting in marginal post-operative improvement. Post-operative refractive measurements on patient datasets expose the inadequacies in using a simplistic model with limited variables. With new devices used in clinical practice the accuracy in measurements of these variables has improved, but the clinical outcomes still vary and are not predictable. Functional vision is an important aspect of post operative outcome not addressed with current preoperative assessments.

Based on clinical studies, several calculations methods have been proposed along with different kinds of regression-based power calculation formulas, and each of these formulas has evolved along with the measurement techniques. However, one of the severe drawbacks of these formulas is that their performance is closely tied to the technology used in the biometry devices. Post-operative refractive outcome prediction rates are better with certain formulas if the biometry data used is from devices employing concepts such as optical low coherence reflectometry instead of ultrasonic waves. The benefit of matching formulae to biometry technology is still inadequate when used across the entire patient population. Along with these variables, various surgical techniques such as incision location based on pre-operative corneal astigmatism and knowledge of posterior astigmatism, lifestyle of the patient before surgery, general and ocular health parameters which has the potential to affect visual performance are some of the pre-operative variables that influence sub-optimal outcomes in a considerable number of patients.

In accordance with the present disclosure, it is believed that post-operative vision compromise is less likely to occur if the pre-operative variables are analyzed not as independent variables, but as a picture which combines the influence of most of the pre- and post-operative measurements. That is, pre-operative measurements, coupled with data on lens tolerances, surgical techniques such incision size and patients' expectation based on their lifestyle before cataract are key to delivering optimal visual outcomes post-operatively. Even today, to determine lens characteristics for an implant, state-of-the-art methods for calculating the correct optical power for an eye are used; most of which are based on linear and non-linear regression methods. However, there is still a high level of uncertainty in clinical outcomes and patient satisfaction. This leaves several patients dissatisfied with the performance of their vision in terms clinically evaluated metrics such as visual acuity, contrast sensitivity and other patient reported outcomes. The cause of visual dissatisfaction can be addressed if the new lens models are used as a choice for patients.

Furthermore, classification of the eyes based on their geometry have revealed that the approximations used in these previously referenced formulas are inadequate and results in poor clinical outcomes especially if the eyes are out of range in terms of power, axial length and several other variables. The regression coefficients used in these formulas serve as scale factors to compensate the inadequacies discussed above. These regression coefficients are highly dependent on the IOL manufacturing tolerances, surgical techniques, pre-operative refractive status and anatomy of the eye of the patient receiving the implant.

With different models of intraocular lens available in the market, the choice of the lens is left to the surgeon and/or patient. Biometry devices and clinical assessments are not capable of taking into account different variables that go beyond the scope of traditional objective measurements, including variability in lens manufacturing, variability in lens design (optical power characteristics, location of axis marks on toric lenses, mechanics of haptics for axial and rotational positioning of the lens), surgeon specific variabilities caused due to variations in incision sizes, surgical techniques and the uncertainty, noise in the biometry devices used to measure the cornea, other optical/geometry characteristics of the patient, and finally the post-operative outcomes from past patients. Some surgeons develop their own site-specific nomograms to account for this variability, but the models are not easy to optimize for visual outcomes. Some of these nomograms are used as empirical factors in power calculation formulas.

In addition to these limitations, it is recognized that the types of feedback desired by surgeons and/or patients may differ at different stages of treatment. For example, during a preoperative stage, recommendations regarding selection of a particular lens or procedure may be desired, alongside predictions of potential success or side effects of the lens and procedure combination. During an operative stage, immediate feedback regarding the procedure employed, the placement of the lens, and the like may be desired by the surgeon. During a postoperative phase, based on results of a procedure, immediate predictive information regarding likelihood of satisfaction with the procedure may be desirable for both the surgeon and the patient.

Accordingly, a platform is provided herein which may be configured to activate and utilize different portions of an overall machine learning model. The model described herein may be a deep learning model, and may be a composite model constructed from a plurality of different models, including other specific deep learning models, classifier models, and the like. In response to different inputs and different stages of treatment, different types of output may be generated from a platform hosting model, for example via an application exposed to and used by the surgeon.

In a particular aspect, as described herein, a learning engine consisting of a training module optimized with different thresholds and activation functions is developed. The custom training module uses different kinds of activation functions depending on the nature of input. A multi-layered perceptron is devised and used as linear classifier for objective measurements and as a binary classifier for subjective measurements such as visual satisfaction, where the patients' reported outcomes are in binary state of either being happy with the surgical outcomes or not happy with the surgical outcomes. The multi-layer perceptron network consists of an input layer, weights or bias, summation and an activation function to trigger the classification of the input into one of the output states.

The use of a model and platform as described herein has a number of advantages over existing systems. For example, use of a neural network approach with a classifier to adaptively learn and generate rules for improving patient satisfaction may improve overall outcomes, as such a system is not limited to analysis of objective refractive error alone, but instead considers overall outcomes.

In addition to the above, when considering use of intraocular lens designs in patients having significant corneal aberrations, it is observed that the presence of higher order aberrations in the cornea and the approximations in the A-constants may, in some instances, offset the benefits of the exact power IOL implantation in the human eye. In eyes with even modest levels of astigmatism, for example, lower image quality on the retina may occur. The detrimental effects caused by presence of any selected aberration in the cornea can be offset by balancing the optical design by introduction of a single aberration, or in some instances, a combination of aberrations. This leads to an IOL design with aberration structure customized based on the spatial frequency content of the image. Using this method developed for designing the aberration balance in an optical element such as an IOL, the image quality can be improved either by balancing any single corneal aberration using a single or a combination of aberrations, or balancing a set of aberrations by a single or a combination of aberrations, in the designed IOL.

As such, and in accordance with aspects of the present disclosure, in some instances an aberration map can be customized based on the individual's corneal aberrations, and a set of images balancing a single or a combination of aberrations may be generated. The cross correlation-based image quality metric provides a tool to perform this balancing between aberrations, and to customize the image quality for any set of aberration(s). As such, in some example embodiments, a method is provided for customizing the aberration map of an intraocular lens based on the corneal topography and spatial frequency content of the image. In some instances, a metric is developed as described herein which provides a direct visualization of the image quality baseline, and helps design the aberration pattern based on the spatial frequency content in the original image instead of basing the optimization on only a single or a restricted set of spatial frequencies.

Any optical system is designed to have best focus and extension in depth of focus when it is used in an optical system consisting of a few other optical elements, in this case the human cornea. IOLs are also designed to provide certain optical characteristics over a certain pupil size. In some instances, current lens design processes may fail to provide the design intent that is appropriate for every patient since lenses are designed for an average population characteristic of cornea and pupil size. Added to this, the corneal power, cylinder and higher order aberrations across human population are not quantized and take a larger range of values than a manufacturer IOLs may typically be designed for.

Because lens models are designed for a specific pupil size, resulting designed lenses cannot achieve comparable optical results for all implanted eyes unless the eye possesses corneal and other optical parameters, such as pupil size, that closely align with the values for which the lens is specifically designed.

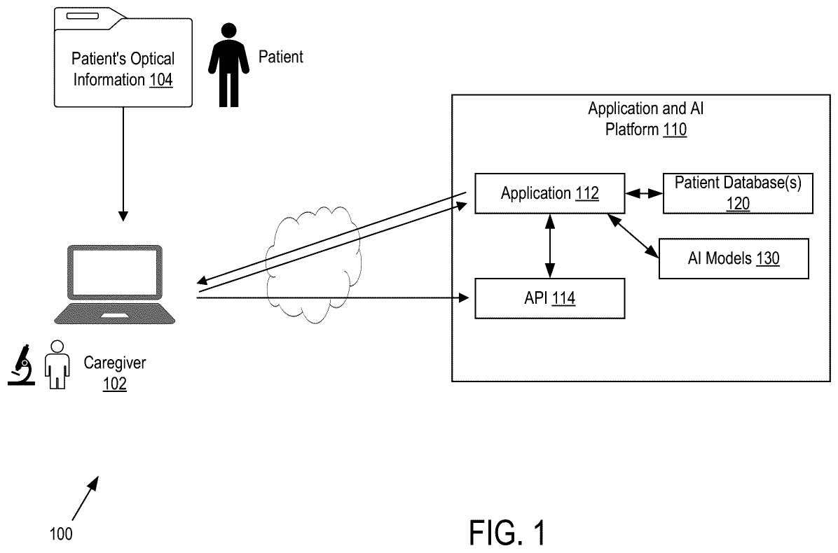

FIG. 1 illustrates an example environment 100 in which aspects of the present disclosure may be implemented. The environment 100 includes a platform 110 that may be interfaced to one or caregiver computing systems 102 via an exposed application 112 (e.g. a web application or the like) or via an API 114. The caregiver computing system 102 may be positioned at a healthcare facility and used by a surgeon or other healthcare personnel to interact with the platform 110, as well as to maintain patient records. In the example shown, the caregiver computing system 102 receives patient optical information 104 of a patient, either as input data or received from external testing equipment, such as optical or ultrasound imaging systems. The patient optical information 104 may be received in any file format, including manual entry or automatically generated from test equipment, and classification schemes may be employed based on use of measurements obtained from either ultrasound measurements or analysis in the electromagnetic spectrum,

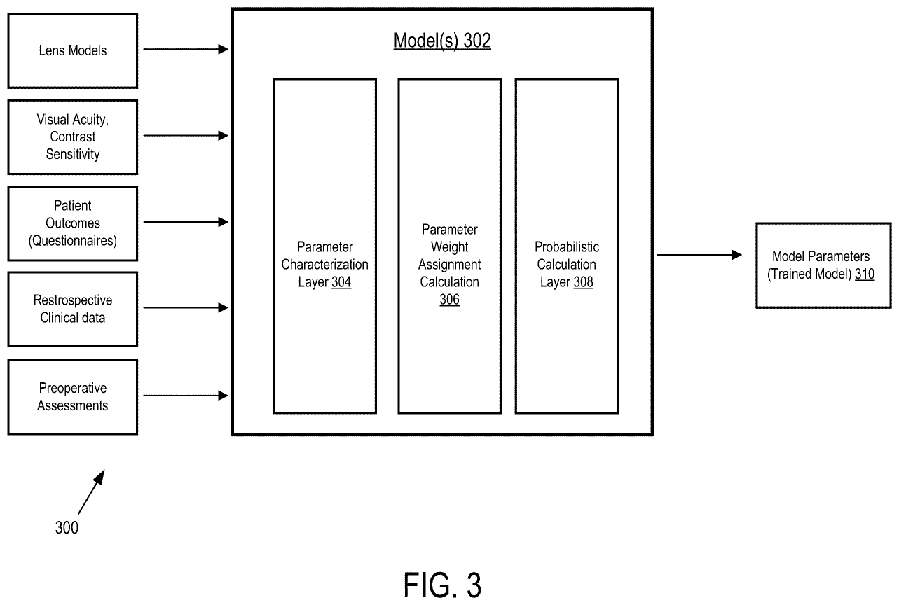

In the example shown, the platform 110 maintains a patient database 120 of received patient optical information 104 from a plurality of different patients, as well as procedure and outcome information from those patients. The platform 110 further implements a machine learning model 130 which may be constructed from one or more deep learning, classifier, Bayesian, or other types of models usable for prediction and guidance as to patient treatment as described herein. Details regarding the application 102, models 130, and patient database 120 containing data usable to train the models 130 are described further below in conjunction with FIGS. 3-6.

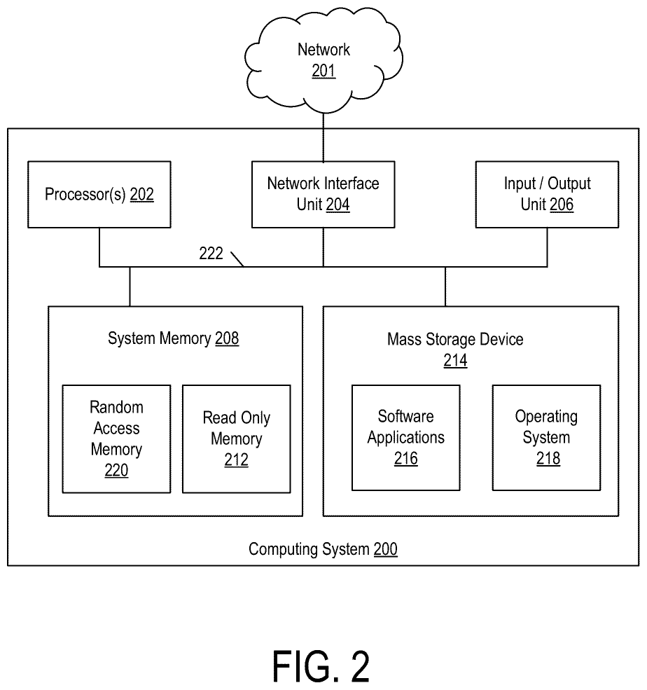

FIG. 2 illustrates an example block diagram of a virtual or physical computing system 200. One or more aspects of the computing system 200 can be used to implement the processes described herein.

In the embodiment shown, the computing system 200 includes one or more processors 202, a system memory 208, and a system bus 222 that couples the system memory 208 to the one or more processors 202. The system memory 208 includes RAM (Random Access Memory) 210 and ROM (Read-Only Memory) 212. A basic input/output system that contains the basic routines that help to transfer information between elements within the computing system 200, such as during startup, is stored in the ROM 212. The computing system 200 further includes a mass storage device 214. The mass storage device 214 is able to store software instructions and data. The one or more processors 202 can be one or more central processing units or other processors.

The mass storage device 214 is connected to the one or more processors 202 through a mass storage controller (not shown) connected to the system bus 222. The mass storage device 214 and its associated computer-readable data storage media provide non-volatile, non-transitory storage for the computing system 200. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, DVD (Digital Versatile Discs), other optical storage media, magnetic cassettes, magnetic tape, magnetic disk storage or other magnetic storage devices, or any other medium which can be used to store the desired information and which can be accessed by the computing system 200.

According to various embodiments of the invention, the computing system 200 may operate in a networked environment using logical connections to remote network devices through the network 201. The network 201 is a computer network, such as an enterprise intranet and/or the Internet. The network 201 can include a LAN, a Wide Area Network (WAN), the Internet, wireless transmission mediums, wired transmission mediums, other networks, and combinations thereof. The computing system 200 may connect to the network 201 through a network interface unit 204 connected to the system bus 222. It should be appreciated that the network interface unit 204 may also be utilized to connect to other types of networks and remote computing systems. The computing system 200 also includes an input/output controller 206 for receiving and processing input from a number of other devices, including a touch user interface display screen, or another type of input device. Similarly, the input/output controller 206 may provide output to a touch user interface display screen or other type of output device.

As mentioned briefly above, the mass storage device 214 and the RAM 210 of the computing system 200 can store software instructions and data. The software instructions include an operating system 218 suitable for controlling the operation of the computing system 200. The mass storage device 214 and/or the RAM 210 also store software instructions, that when executed by the one or more processors 202, cause one or more of the systems, devices, or components described herein to provide functionality described herein. For example, the mass storage device 214 and/or the RAM 210 can store software instructions that, when executed by the one or more processors 202, cause the computing system 200 to receive and execute managing network access control and build system processes.

In examples, the disclosed computing system provides a physical environment within which aspects of the present disclosure may be implemented. For example, the computing system may represent a computing system with which the platform 110 may be implemented and on which a deep-learning model be trained or be used for inference, or a computing system on which the data set is generated.

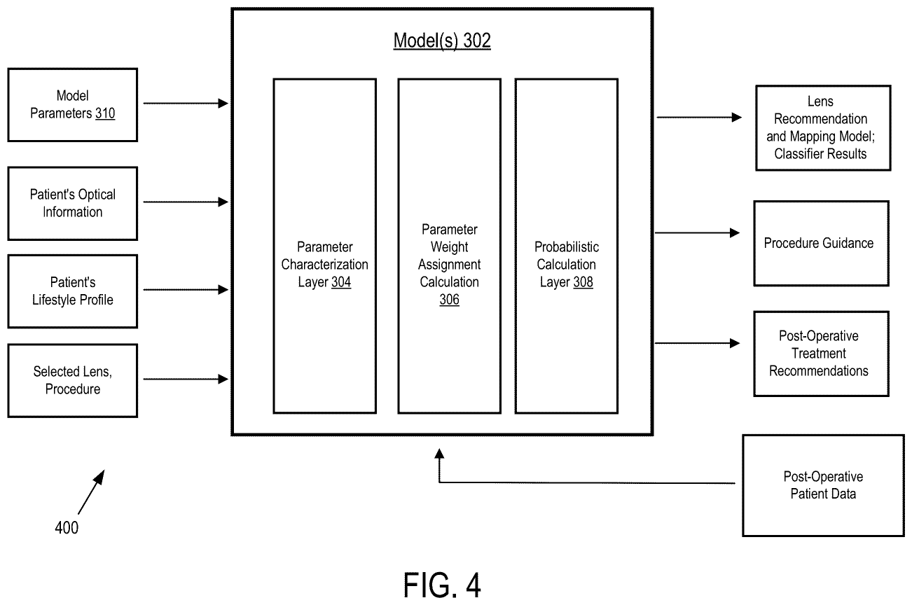

Now referring to FIGS. 3-4, training and inference processes regarding an example machine learning model implemented as one or more of models 130 hosted by platform 110 is provided. In particular, FIG. 3 illustrates a training process 300 for a machine learning model 302, such as may be hosted by an ophthalmic treatment recommendation and guidance platform, and useable to generate recommendations for treatment in accordance with example aspects of the present disclosure.

In the example shown, the model 302 may include one or more of a perimeter characterization layer 304, a parameter weight assignment calculation 306, and a probabilistic calculation layer 308. In the example as illustrated, the model 302 may receive various types of information as training data. Such training data may include lens model information, such as manufacturer lens power data, lens modulation transfer function measured with one or more corneas, modulation transfer function of the lens, lens geometry including but not limited to anterior radius, posterior radius, thickness and refractive index, mechanical description of the lens such as compression force and haptic angle, clinically measured lens rotation data, axial location, tilt, decentration, and the like. The training data may also include preoperative assessment data, including measurements of a patient's eye, including patient's optical corneal data, anterior chamber depth, axial length, white to white distance, phakic lens geometry, capsular bag size, and the like. The training data may also include procedural information regarding historical procedures and outcomes, such as surgical accessories used to create the incision and incision location, refraction measurement visual satisfaction attributes for lens selection, surgical process, choosing between optical and ultrasound biometry, cataract grade of the lens, environmental parameters including but not limited to pressure, temperature and humidity of the surgical site or suite, and the like. The training data may include other patient assessment information, such as clinical measurements of visual acuity and contrast sensitivity under different lighting conditions, reading performance under a clinical or real world settings. Other patient data may also be used, including patient subjective data such as patient's visual effects questionnaire, a life style questionnaire (e.g., driving, reading, day/night habits and preferences and the like) as well as, ocular and general health history, etc.

During a training process, all of this information may be captured as associated with a large number of historical patients. Once trained, the models 302 may be configured with a plurality of model parameters, representing a trained model (seen as model parameters 310).

Once trained, the model parameters 310 may be implemented in a production version of the models 302. In addition, as seen in the prediction and guidance process 400 of FIG. 4, a variety of information associated with a particular patient may be provided to the models. The specific information provided may differ based on the individual patient and stage of treatment. In some instances, a patient's optical information and lifestyle profile may be received. The optical information may include entered or measured eye characteristics, analogous to the training data described above. Optionally, one or more selected lenses and procedures may be provided to the model 302 as well, as candidate lenses and procedures for use by the surgeon.

In the recommendation phase, the model 302 may output one or more types of information predicted from the training data. In some examples, a lens recommendation and mapping model may be output, with particular lenses associated with particular procedures and likelihoods of success. In some implementations, each input candidate lens may be associated with a best candidate procedure and likelihood of success, and that full set of lens, procedure, and success rate may be output by the model. In alternative implementations, only a best candidate lends, procedure, and likelihood of success may be output. As described further below, in some instances the model may be used during an operative phase or postoperative phase as well. In such instances, the model 302 may be configured to generate procedure guidance regarding incision location and/or depth and the like. In response to the procedure as performed, the model 302 may receive procedural information from the surgeon and may output one or more postoperative treatment recommendations to maximize success, reduce recovery time, and otherwise improve outcomes.

Referring to the models 302 specifically, it is noted that every input data set to be used for post-operative predictions and hence pre-operative adjustments are trained and weighed by the modules, and characterization parameters are adjusted adaptively with every new patient's data. The results obtained for different parameters are tied to another algorithm based on the Bayesian approach and are weighted depending on the noise in each variable's classification scheme. Within these inputs to the AI system are several subgroups of data inputs such as deep learning, supervised and unsupervised learning, classification, translation, text generation, answering questions, and signal recognition, which includes audio and video. The training/learning and decision system is built as a combination of a learning and a concept rich system, capable of using output from learning and apply different concepts to make a consistent decision, and continuously improve the decision by monitoring for abnormal behaviors and outliers by seeking information from every new patient's data.

The network used for the models 302 may be implemented using a layered structure with the layers represented by input data, output data and an intermediate layer which converts input data or pattern to output data or pattern. In the case of clinical outcome predictions, the input data is a set of objective data such as measurements, degree of training to operate the devices to make measurements, subjective inputs from patient profile such as occupation, driving habits and so on. The network is trained, weights are calculated and a probabilistic calculation based on distance measure within the training data helps to estimate confidence in classification. These distances are adaptively changed depending on the accuracy of the postoperative outcomes.

In some implementations, the models 302 are designed to decide between “n” different optical designs based on “m” items of clinical information, there will be “m” input data points mapped into “n” output data points. The number “n” is adaptive and the system trains and learns to adapt to changing values of “m” and “n.” As noted previously, the models 302 are designed to pick the optimal design from the “n” output data points based on a combined probability calculation weighed by the confidence in probability calculations, which is also adaptive. One or more best candidate designs may be provided to the surgeon for selection.

In accordance with the modeling approach described herein, a success or failure for not only the overall procedure, but for every variable may be calculated based on the distance of the calculated probability with reference to a prior discriminant. For post-operative outputs such as visual satisfaction, an illustration will be a 2-level adaptive branch prediction with branch outcomes of +1 and −1. Training using the data finds correlations between history and outcomes. An initial guess weight is given for every input “xi” and mapped into a single output “y” such that the output y=w₀+−Σᵢ₌₁ⁿxᵢ kᵢ. Based on the distance of the output from the boundaries of the training data, the probability of the output being true or false is estimated. In one operative example, let pi be the probability of the input i. In this, i denotes different inputs such as corneal keratometry, anterior chamber depth and all other variables including site and manufacturer specific variables. For a given site, the manufacturer-based variables are treated separately to enable the learning/training/prediction engine to make a judgment on the lens model to be implanted, independent of the manufacturer but factoring in the manufacturer bias once the patient specific data estimate is completed.

In case the data is not a representation of the actual state of the model, an illustration when a surgeon has been replaced by another surgeon or a clinical associate has been replaced by a different clinician is used. A Hidden Markov model is used in such cases to improve probability of predictability.

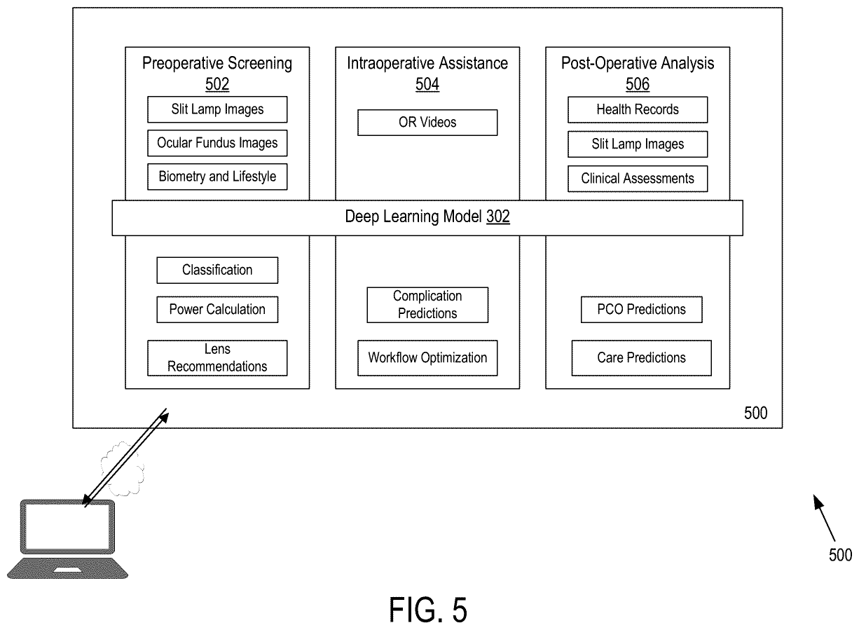

FIG. 5 illustrates a logical diagram of an application 500 that uses the machine learning model of FIGS. 3-4 to generate treatment recommendations and predictions, according to example aspects of the present disclosure. The application 500 may represent an example of application 120 described above in conjunction with FIG. 1, and usable with models 130, 302 described herein. In the example shown, the application 500 includes a preoperative screening phase 502, and intraoperative assistance phase 504, and a postoperative analysis phase 506.

The preoperative screening phase 502 may be used by a surgeon to provide inputs regarding a patient prior to initiation of treatment. The inputs may include one or more slit lamp images, ocular fundus images, and biometry measurements of the patient. The inputs may also include one or more lifestyle factors, such as driving, night vision, and the like. Based on the specific inputs provided during the preoperative screening phase 502, a model, such as deep learning model 302 may generate predictions, or recommendations, regarding treatment as described above. For example, a classification of the patient and potential procedures to be performed may be provided as well as a power calculation for a desired lens to be used during surgery. One or more lens recommendations may also be generated during the preoperative screening phase 502, as well as likelihood of success as to particular conditions or overall success (as measured by general patient satisfaction).

In some examples, the preoperative screening phase 502 may implement a customized classification scheme and pre-operative decision and lens parameter adjustment for ophthalmic lenses and related visual devices from any number of measurements. These measurements may include objective measurements from the manufacturer such as power. The model 302 may generate a classification in accordance with the likelihood associated with that parameter yᵥ=k₀+Σᵢ₌₁ⁿxᵢ kᵢ; yv=−1 for yv<0 and yv=+1 for yv>0 where “v” is the input variable and in case of continuous variables where the values of yv are non-binary, calculates the probability of classification within the set of defined classifiers; a combined probability is used for decision making by an artificial intelligence system, such that the correlating weights ki is proportional to the probability that the outcome predicted branch lies within the classes −1 or +1 or an analogous value x; supervised training procedures are employed with the error-correction learning rule as the basis for back-propagation.

In some instances, the specific classifiers used in generating a lens recommendation or procedure recommendation may include all classifier models available. In other implementations, recommendations may be made either by using all the classifiers, or by automatically removing classifiers based on the level of confidence in any classifier.

The intraoperative assistance phase 504 may receive operating room videos or feedback regarding an operative procedure that is performed on the patient. The models, such as model 302 may generate one or more workflow optimizations as well as complication predictions based on observed events during the procedure.

The postoperative analysis phase 506 may receive health records of the patient including operation records and any preoperative screening data, as well as postoperative slit lamp images and clinical assessments. Based on this information, the models 302 may generate primary care predictions and other care predictions regarding likely outcomes or subsequent treatments that may be advisable following surgery.

Referring to FIGS. 3-5 generally, it is noted that the application 500 may generate different recommendations or outputs based on the information provided to the models 302. This may be due to the application 500 calling different ones of the models, or based on the model selectively activating different classifiers or predictors in response to receipt of particular information. In some cases, the modeling and application may be configured to make decisions using some or all classifiers included in the overall model. For example, all the classifiers may be used, or fewer than all classifiers may be used, with classifiers being removed based on the level of confidence in any classifier

$$P ( {q}_{1} , {q}_{2} , {q}_{3} … {q}_{t} \vert {o}_{1} , {o}_{2} , {o}_{3} … {o}_{t} ) = \frac{P ( {o}_{1} , {o}_{2} , {o}_{3} … {o}_{t} \vert {q}_{1} , {q}_{2} , {q}_{3} … {q}_{t} ) P ( {q}_{1} , {q}_{2} , {q}_{3} … {q}_{t} )}{P ( {o}_{1} , {o}_{2} , {o}_{3} … {o}_{t} )} = 0$$

or 1 based on preset threshold value of probability.

Classifiers, also referred to herein or useable as training data, may include manufacturer lens power data, lens modulation transfer function measured with one or more corneas, modulation transfer function of the lens, lens geometry including but not limited to anterior radius, posterior radius, thickness and refractive index, mechanical description of the lens such as compression force and haptic angle, clinically measured lens rotation data, axial location, tilt, decentration, patient's optical corneal data, anterior chamber depth, axial length, white to white distance, phakic lens geometry, capsular bag size, surgical accessories used to create the incision and incision location, refraction measurement visual satisfaction attributes for lens selection, surgical process, choosing between optical and ultrasound biometry, cataract grade of the lens, environmental parameters including but not limited to pressure, temperature and humidity of the surgical site or suite, clinical measurements of visual acuity and contrast sensitivity under different lighting conditions, reading performance under a clinical or real world setting, patient's visual effects questionnaire, life style questionnaire, ocular and general health history, delivery system and viscoelastic during surgery.

Furthermore, in example implementations, because patient, surgeon, and other data is all provided to classifier models included in the models 302, the model predictions that are made may be surgical site specific, patient specific, device manufacturer specific and surgeon specific. Additionally, the number of classifications adaptively increases as the number of variables increase and the scheme has the capability to differentiate independent variables and create new classification such that f(x)=−1 if x<0 and f(x)=1 if x≥0 and f(x) is some analog value for multiple classification based on available lens models, non-linear activation method is chosen.

As noted herein, the models that are used may be adapted and retrained in at least near-realtime. Such adaptability may improve reliability and fine tuning of recommendations, for example, by computing rate of change of weights for each variable and assigning weighting for probability of each variable based on distances

$$\frac{\vert {y}_{v} - {C}_{- 1} \vert}{\vert {C}_{- 1} - {C}_{+ 1} \vert} and \frac{\vert {y}_{v} - {C}_{+ 1} \vert}{\vert {C}_{- 1} - {C}_{+ 1} \vert}$$

and probability multipliers scaled based on a preset threshold.

In some implementations, the models 302 may be trained using various data, including objective measurements from the manufacturer such as power and attributes from patient's lifestyle and tasks questionnaires and surgeon's notes, classifies the likelihood associated with that parameter and calculates the probability of classification within the set of defined classifiers such that the outcome with the current data based on nature of the stochastic process and apply Markov models on the patient outcome and is based on the dependencies of current information with previous information such that P(Oₙ)=Πᵢ₌₁ⁿP (Oᵢ|Oᵢ₋₁) where (Oₙ) is the outcome for the current patient based on the outcome Oᵢ₋₁ of patient i−1. In such instances, the model may be trained iteratively until a stop time. The stop time may be defined as the period at which the variability in the individual perceptron weight has stabilized for that patient after a period of site training or the surgeon trained in particular surgical implant; if O={o1, o2, o3 . . . ot} where oiε{good outcome, bad outcome}; each observation comes from an unknown state and will also have an unknown sequence Q={q1, q2, q3 . . . qt} where qiε{monofocal, extended depth of focus, bifocal, trifocal, quadrifocal or any lens models or variations within any model to account for toricity or aberration profile}, to calculate

$$P ( {q}_{1} , {q}_{2} , {q}_{3} … {q}_{t} \vert {o}_{1} , {o}_{2} , {o}_{3} … {o}_{t} ) = \frac{P ( {o}_{1} , {o}_{2} , {o}_{3} … {o}_{t} \vert {q}_{1} , {q}_{2} , {q}_{3} … {q}_{t} ) P ( {q}_{1} , {q}_{2} , {q}_{3} … {q}_{t} )}{P ( {o}_{1} , {o}_{2} , {o}_{3} … {o}_{t} )}$$

which is a combined probability used for decision making by an artificial intelligence system for patient k such that

$$Pk = = \frac{\vert {y}_{v} - {C}_{n} \vert}{\vert {C}_{n} - {C}_{m} \vert} = - 1$$

for Pk<0 and +1 for Pk>0 and is not used in the decision-making process if Pk=0.

In example implementations, the application 500 is capable of calculating and using optical characteristics such as lens spherical power, toric power and aberrations and has the capability to learn and compensate for changes in lens positions without the dependence on any manufacturer provided intraocular calculator constants including but not limited to A constant.

Furthermore, the application 500 may use optical or ultrasound technology in low dense cataract patient and use estimated differences in vitreoretinal interface as a classifier in patients with high dense cataract. This is input to the deep learning algorithms (e.g., models 302) to predict a corrected axial length. The difference in location of vitreoretinal interface between ultrasound and optical technique from cross correlating optical surface locations may also be used. The learning from this is used to estimate the vitreoretinal interface with higher accuracy in patients in dense cataract and optically compromised ocular media. Furthermore, the mechanical characteristics during the surgical procedure may be estimated to mitigate risk of posterior capsular rupture during surgery.

Overall, the learning and training parameters used in FIGS. 3-5 increase prediction reliability of post operative outcomes in patients with high axial myopia, poor fixation and dense media opacity. The optical characterization parameters such as modulation transfer function (MTF) and aberration profile of intraocular lenses at different pupil sizes combined with the optical measurements of the patient's eye may be used to predict clinical outcomes including but not limited to visual acuity and contrast sensitivity under different lighting conditions. Various visual disturbances may be determined preoperatively, for example through focus MTF measurements of intraocular lenses (IOLs) alongside retrospective clinical data.

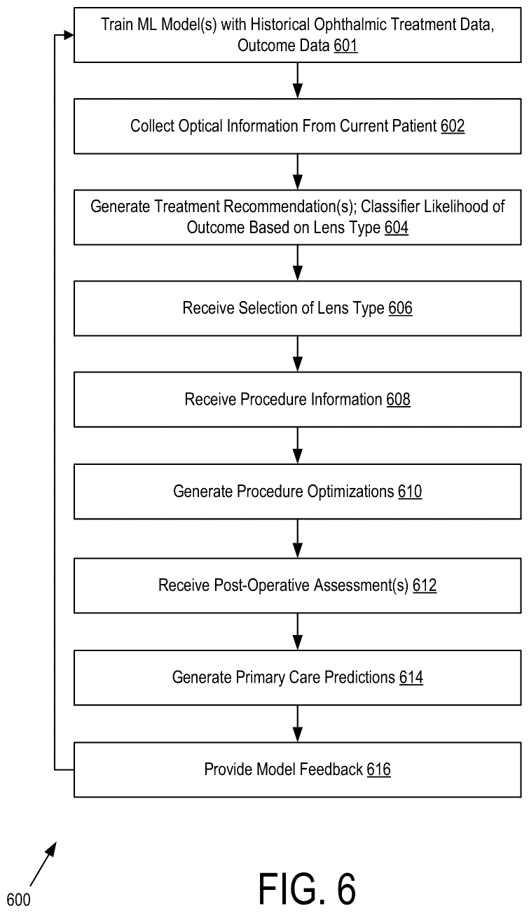

FIG. 6 illustrates an example method 600 of use of the ophthalmic treatment recommendation and guidance platform described herein. The method 600 may be implemented using the models and platform described above. In examples, the method 600 may be performed using an application 112 usable to interface with one or more models 130, 302 as described herein.

In the example shown, the method 600 includes training the one or more machine learning models with historical ophthalmic treatment data associated with a plurality of patients (step 601). The historic ophthalmic treatment data may include patient data, procedure data, lenses used, and outcome information in the form of patient satisfaction. Various other training data is described above in conjunction with FIG. 3.

In some examples, patient feedback information may be obtained in the form of responses to questionnaires that are directed to patient subjective vision considerations, for example the extent a patient has difficulty reading ordinary printing newspapers when wearing glasses or contacts, difficulty in noticing objects in peripheral vision, and the like.

The method 600 further includes collecting optical information from a current patient (step 602). The optical information can include, for example, the optical information includes one or more input parameters including eye dimensional measurements, as well as candidate lenses and treatments for which potentiality for success is to be assessed.

In the example shown, the method 600 further includes generation of treatment recommendations (step 604). The treatment recommendations may each be associated with a likelihood of a particular outcome based on a lens type and treatment plan. As noted above, either a best candidate lends and treatment plan may be displayed, or separate likelihoods of outcomes may be displayed in association with different lenses and treatment plans to allow for selection by a surgeon in consultation with the patient.

In the example shown, the method 600 includes receiving selection of a lens type from among the recommendations presented (step 606). The selection may include a selection of a treatment plan or procedure to be used in conjunction with the lens type. The method 600 may also include receiving procedure information, for example during an operative phase of treatment (step 608). In response, the method 600 may include generation of one or more procedure optimizations during the operative phase (step 610). This may be performed, for example by generating, at the deep-learning model, one or more optimizations for use during the ophthalmic procedure, the one or more optimizations being presented to a caregiver via an application exposed by the ophthalmic treatment platform.

In the example shown, the method further includes receiving one or more postoperative assessments (step 612). The one or more postoperative assessments may include a surgical assessment provided by the surgeon or caregiver team, such as an assessment of the procedure as performed, tracking of side effects of the procedure or irregularities, and the like. The postoperative assessments may further include self-assessment by the patient, for example subjective vision assessment immediately after surgery, and after a period of time thereafter. In response, the method 600 may further include generating one or more primary care predictions (step 614). The primary care predictions may correspond with recommendations regarding recuperation and follow-up care that may be advisable for the patient following surgery.

The method 600 further includes providing model feedback (step 616). Providing model feedback corresponds to collection of patient data, including the patient information, procedure information, lens information, and outcome information, and providing all of that to the machine learning model for retraining, thereby ensuring continuous improvement and adaptability of the model to account for new lenses, procedures, and the like, and continually generate improved predictions and recommendations. It is noted that, although providing model feedback is described as being performed upon completion of treatment, the systems and methods described herein, including the platform 110 as implemented, may be constructed to perform continual retraining processes. For example, information such as the propensity for patients to select a particular lens type, the propensity for the system to generate particular procedural optimizations, and the use of such optimizations, may be used prior to completion of treatment of a patient as part of training data for subsequent patients.

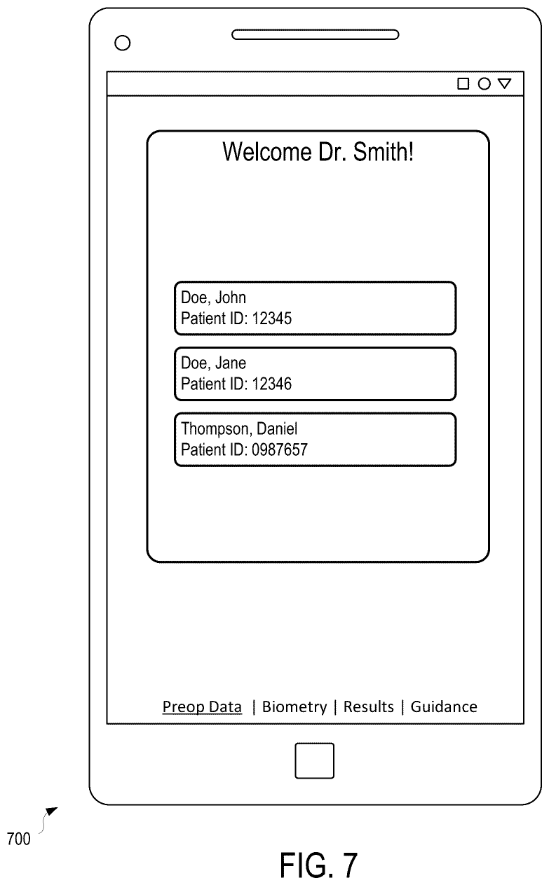

Referring to FIGS. 7-13, a series of user interfaces are depicted. The user interfaces may be presented on a caregiver computing system, such as caregiver computing system 102 described above, in response to interaction with the ophthalmic treatment recommendation and guidance platform, according to an example embodiment. In some examples, the user interfaces may be generated by an application, such as application 120 described above.

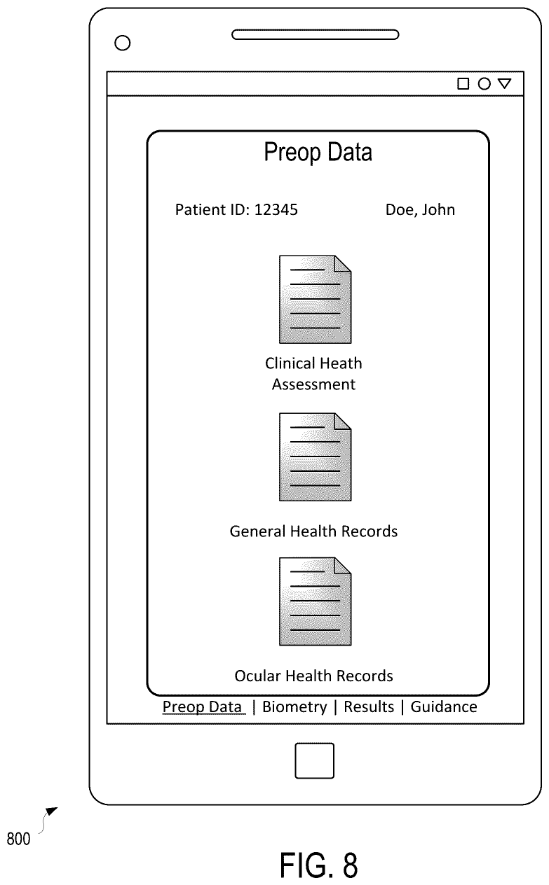

FIG. 7 illustrates an initial patient data user interface 700 usable by a caregiver, such as a surgeon or other healthcare provider, to select a particular patient having a patient record associated with that caregiver. As illustrated in FIG. 8, a further user interface 800 may be presented upon selection of a particular patient. Within the user interface 800, the caregiver may select from among various patient records, such as a clinical health assessment, general health records, and ocular health records.

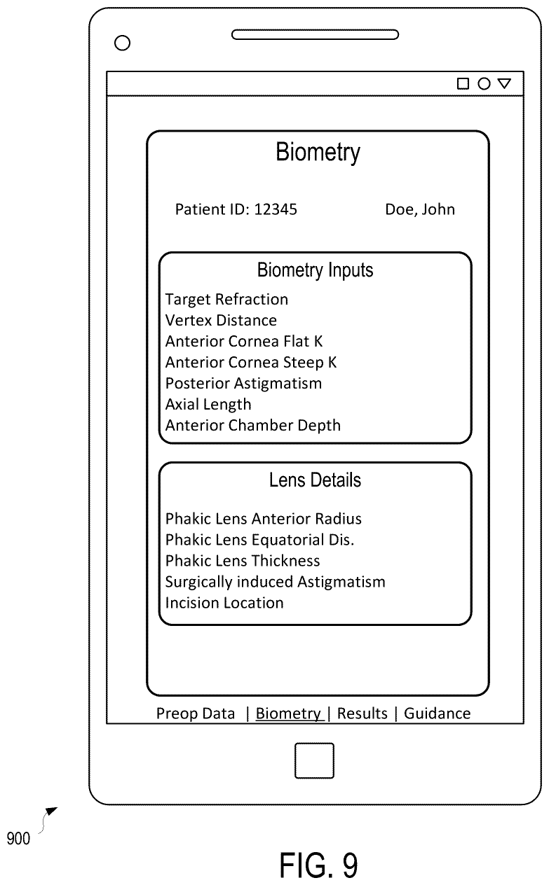

If a user of the caregiver computing system 102 selects a “biometry” option within the user interface, a biometry user interface 900 may be presented, as seen in FIG. 9. Within the biometry user interface 900, the caregiver may enter various biometry inputs and or view such inputs associated with a patient. Biometry inputs may include a target refraction value, a vortex distance, and anterior cornea flat K value, an anterior cornea steep K value, a posterior astigmatism value, and axial length, and an anterior chamber depth. Other values may be input or viewable as well. In addition, various lens details may be presented for a lens to be applied or which has been applied to the patient. Such lens details may include specific radius, distance, and thickness of a lens, any surgically induced astigmatism, and an incision location at which the lens is positioned.

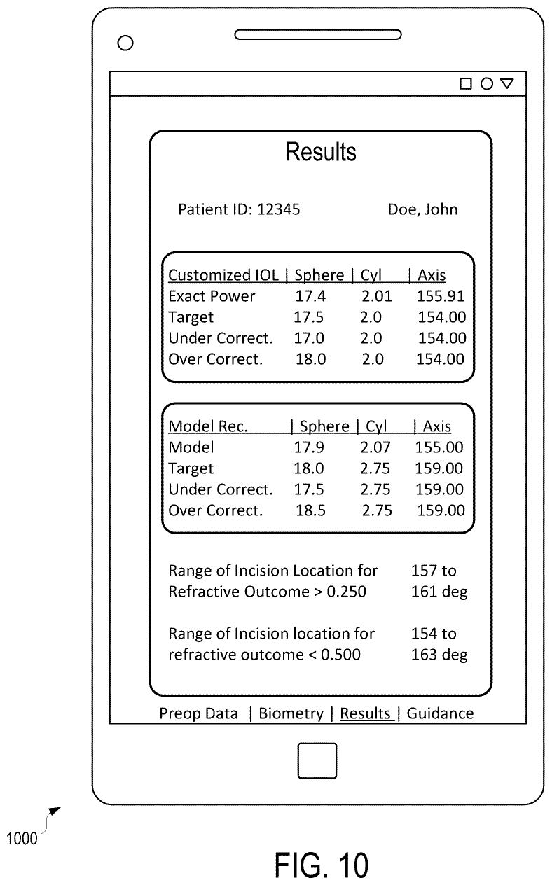

FIG. 10 illustrates a results user interface 1000 presented on the caregiver computing system based on interaction with an application hosted by the ophthalmic treatment recommendation and guidance platform, according to an example embodiment. In this example, the results user interface 1000 may present output results of a trained model, such as models 130, 302 described above based on the patient data received at the application and provided to the trained model. In particular, in the results user interface 1000, optical power targets are generated for a particular patient, as well as a target location or range of locations for an incision for surgically introducing an intraocular lens (IOL).

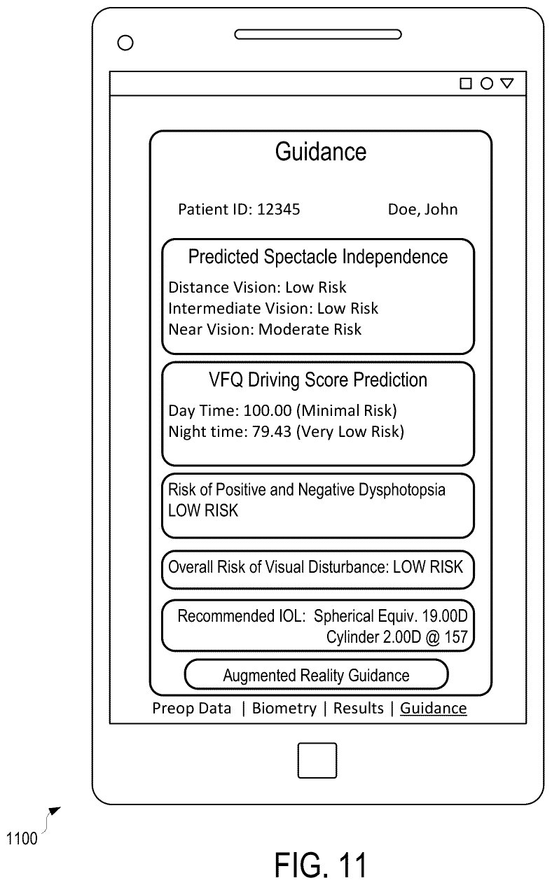

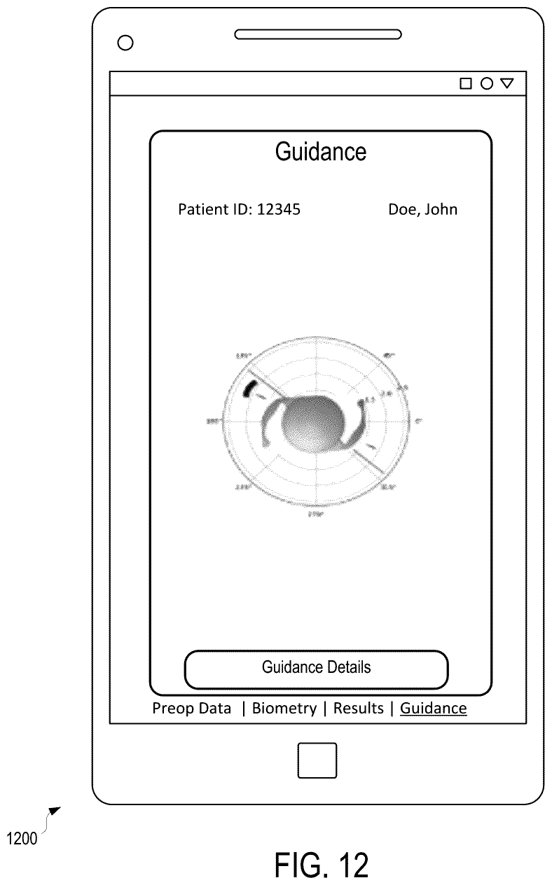

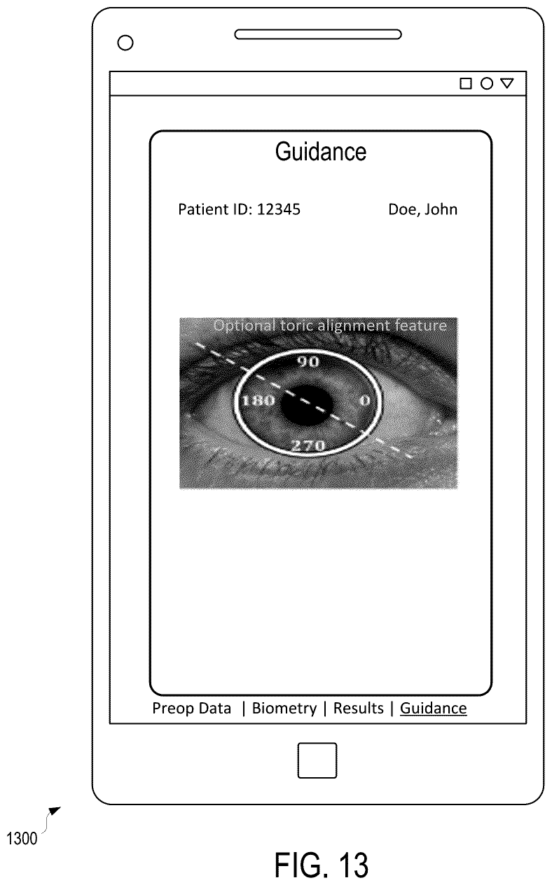

FIGS. 11-13 illustrate example guidance user interfaces that may be generated from models 130, 302 and which provide information regarding operative procedures and guidance to the caregiver and/or patient regarding expected outcomes, potential risks, and confidence values in those outcomes/risk levels. In particular, in the example guidance user interface 1100 shown in FIG. 11, predicted postoperative outcomes are depicted for the patient given the recommended lens and procedure to be used. For example, a prediction regarding spectacle independence, driving score for daytime and nighttime driving, and risks of other visual disturbances may be rated according to high, medium, or low risk. An augmented reality guidance option may be selected, in which case one or more user interfaces 1200, 1300 may be presented which depict either schematic illustrations of surgical procedures to be performed on the patient including particular graphical illustrations of incisions to be used (in FIG. 12), or an augmented reality depiction of toric alignment (in FIG. 13)

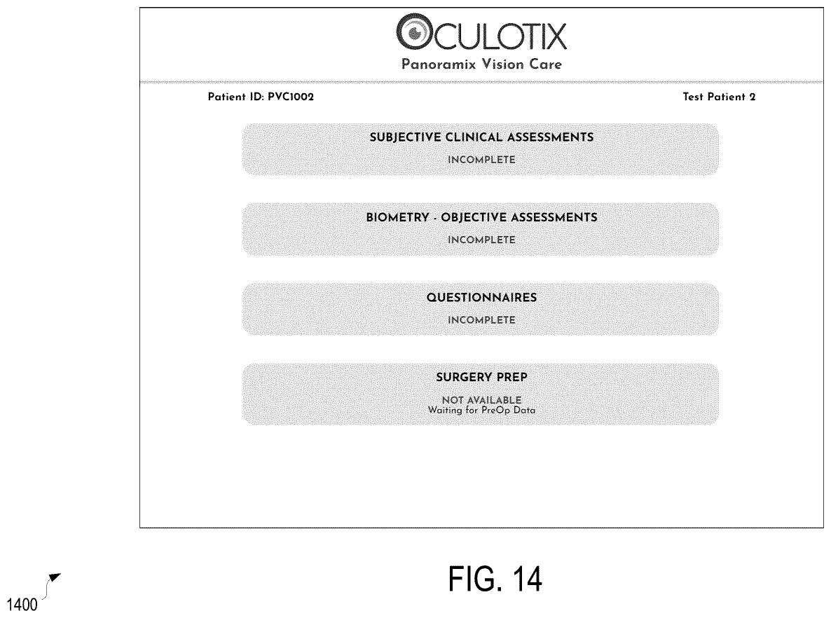

Referring to FIGS. 14-26, a further set of user interfaces are illustrated. The user interfaces 1400-2600 of FIGS. 14-26, respectively, collect and present various other patient information gathering, surgical outcome prediction, and surgical guidance features. The user interfaces described here may be presented on a desktop or tablet computing system, for example, as compared to the mobile device illustrated in FIGS. 7-13; however, it is noted that the same information may be presented on caregiver computing devices of various formats.

FIG. 14 illustrates a patient data user interface 1400 presented on a caregiver computing system, such as caregiver computing system 102. The patient data user interface 1400 depicts a status of patient data collection, including subjective clinical assessment, objective assessments of patient biometry, patient questionnaires, and ultimately surgical preparation data.

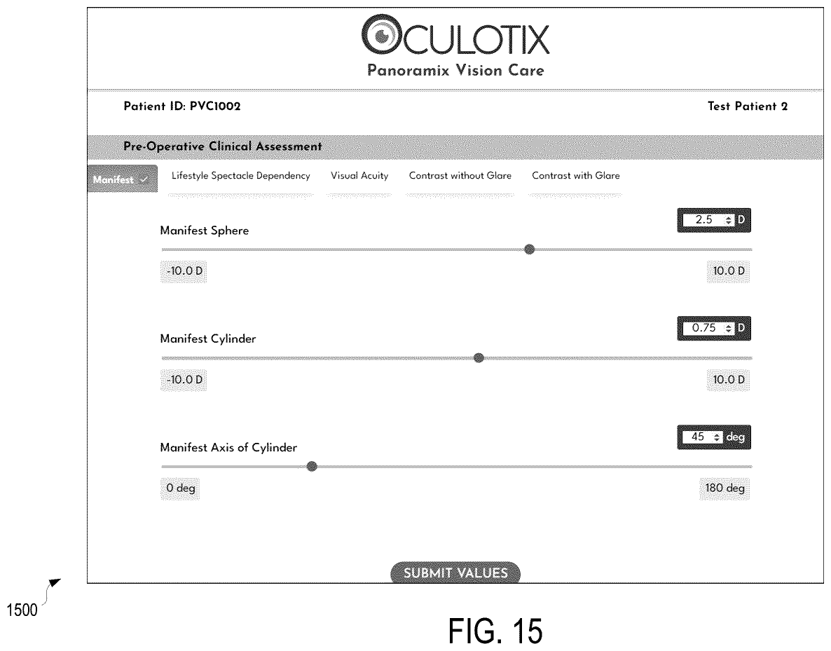

FIG. 15 illustrates a clinical assessment user interface 1500 presented on a caregiver computing system, such as caregiver computing system 102. The clinical assessment user interface 1500 may be presented in response to selection of the subjective clinical assessment option within the patient data user interface 1400. The clinical assessment user interface 1500 may receive a variety of data values, including a manifest ear value, a manifest cylinder value, a manifest axis of cylinder value, and the like. Other types of information, such as life style spectacle dependency, visual acuity, contrast with and without glare may be input as well.

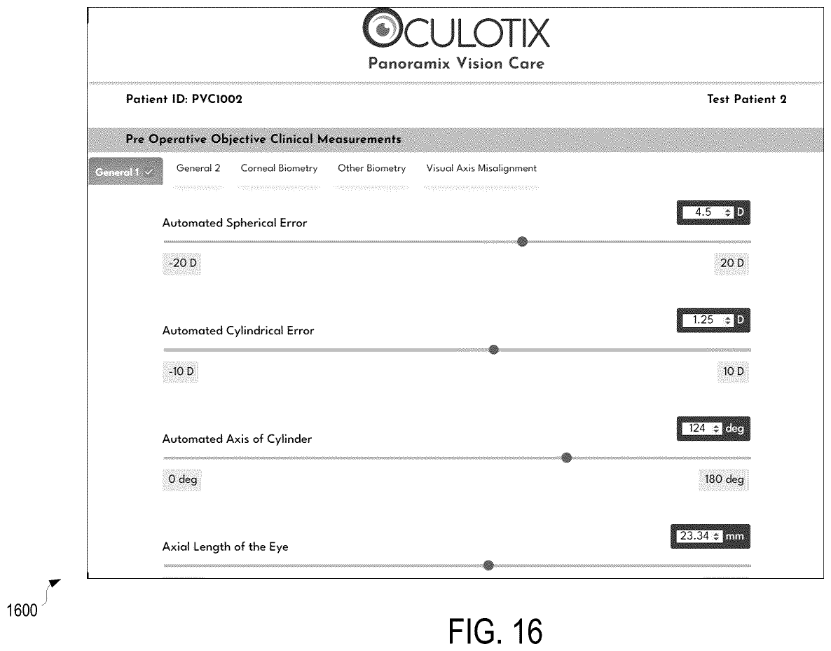

FIG. 16 illustrates a preoperative clinical measurement user interface 1600 presented on a caregiver computing system, such as caregiver computing system 102. The clinical measurement user interface 1600 may receive entry of a plurality of data values including automated spherical error, automated cylindrical error, automated axis of cylinder, axial length of the eye, and the like. Additionally, corneal biometry, visual axis misalignment, and various other biometry measurements may be obtained and entered as well.

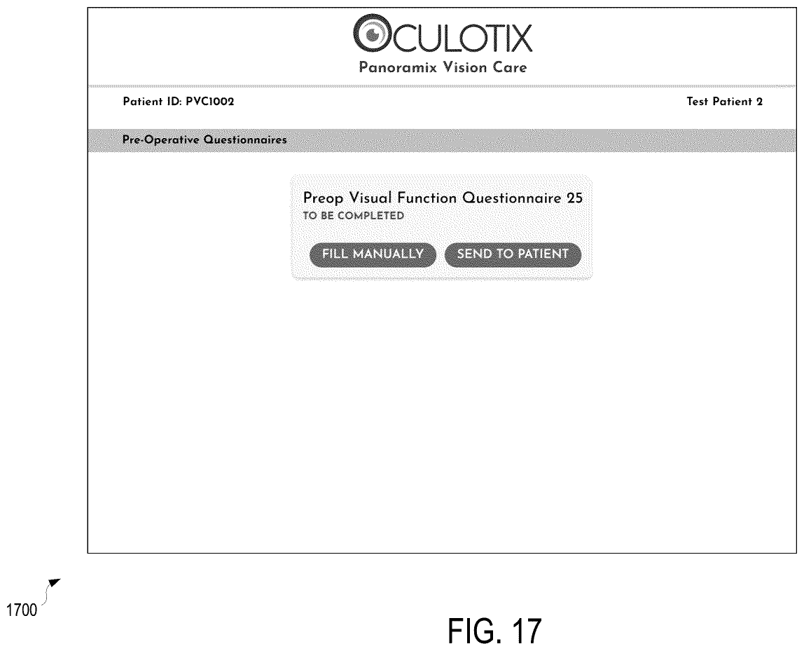

FIG. 17 illustrates a questionnaire user interface 1700 presented on a caregiver computing system, such as caregiver computing system 102. The questionnaire user interface 1700 may lead a user to a series of questions regarding visual preferences and experience to obtain a patient user's subjective interpretation of desirable outcomes for surgery.

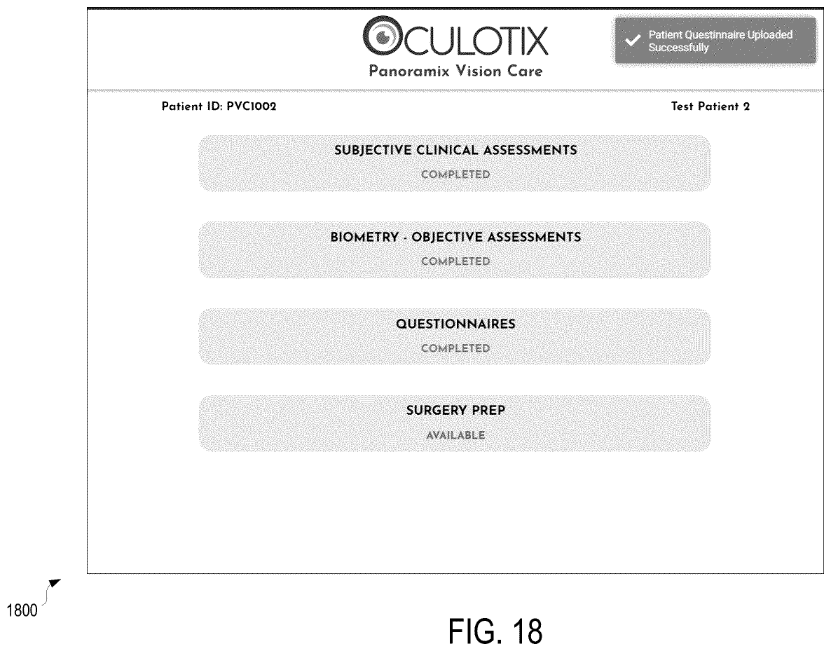

As seen in FIG. 18, once all of the information from the user interfaces 1500-1700 is collected, a patient data gathering status user interface 1800 may be depicted. The patient data gathering status user interface 1800 may correspond to the patient data user interface 1400, there may be enabled a surgery prep option allowing a caregiver to view surgery preparation recommendations and enter settings.

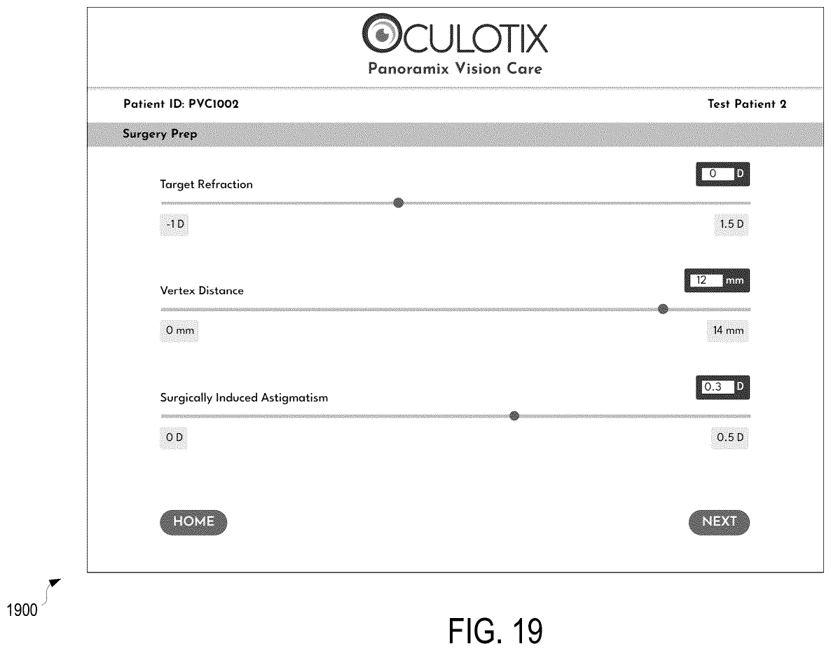

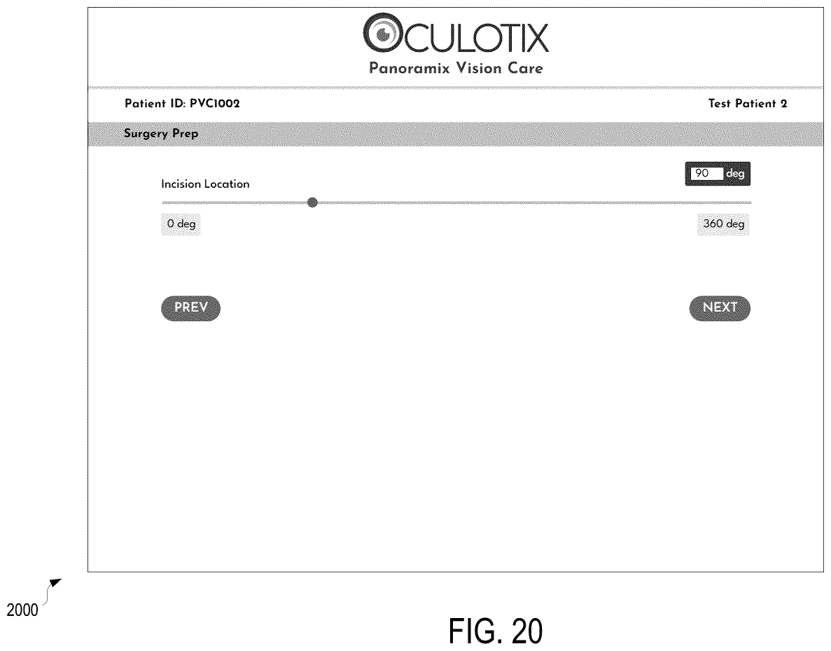

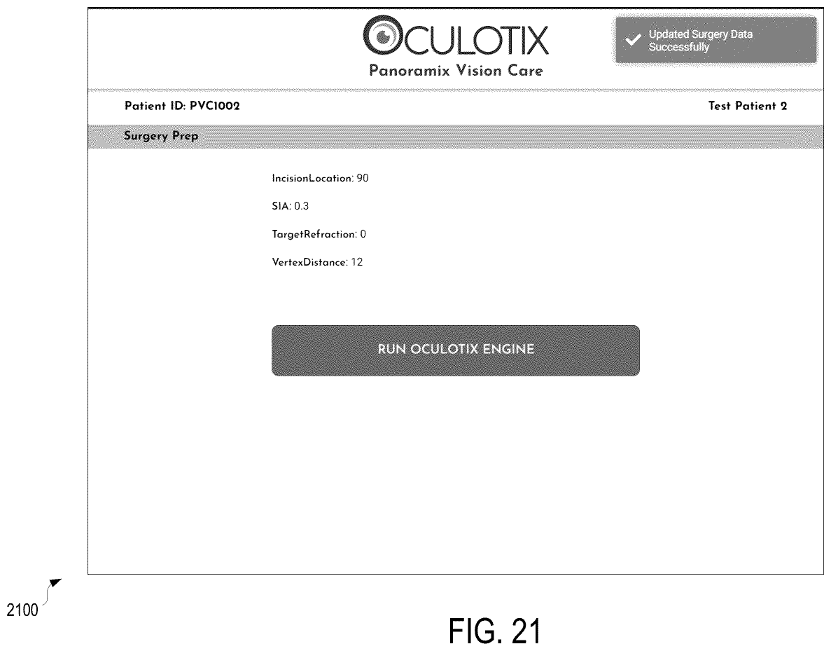

FIG. 19 illustrates a surgery preparation user interface 1900 presented on a caregiver computing system, such as caregiver computing system 102. The surgery preparation user interface 1900 may be presented in response to selection of the surgery prep option within the patient to data gathering status user interface 1800 described above. In the example shown, a plurality of data values may be entered, such as target refraction values, a vertex distance, and a surgically induced astigmatism. Additionally, as seen in FIG. 20, a further surgery preparation user interface 2000 may receive incision location values. Once all surgery preparation values are entered, surgery preparation user interface 2100 of FIG. 21 may be depicted. The user interface 2100 may present the surgery values for confirmation, and receive an input to run a recommendation engine as described herein.

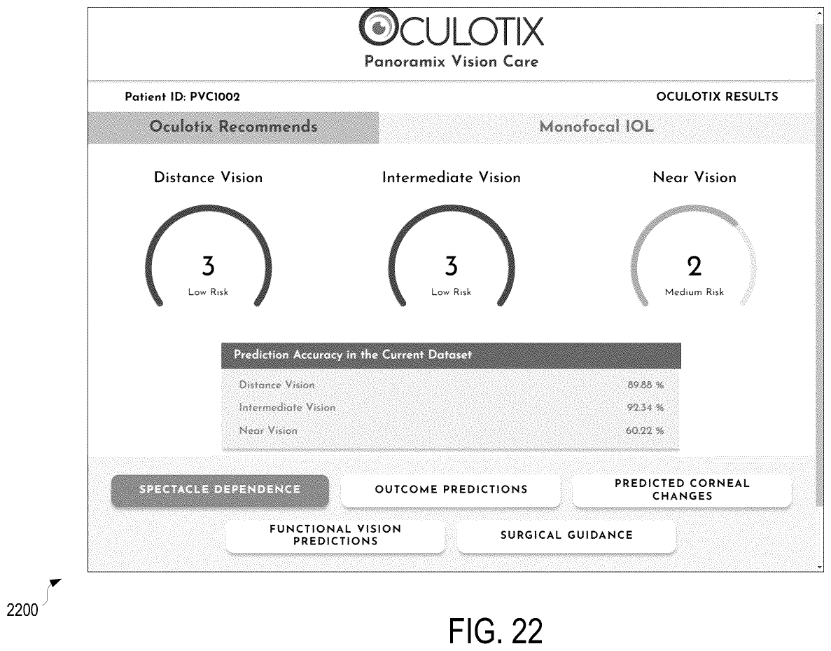

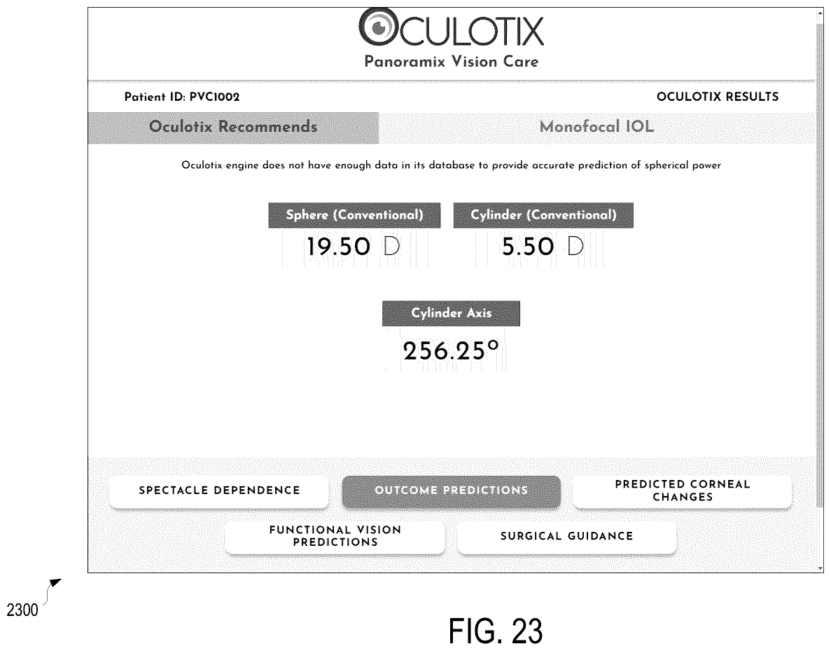

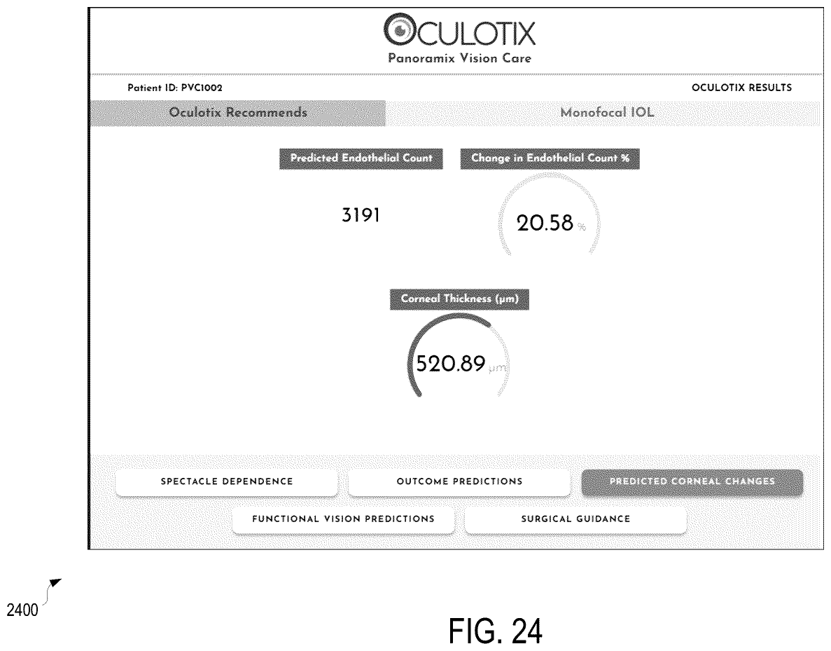

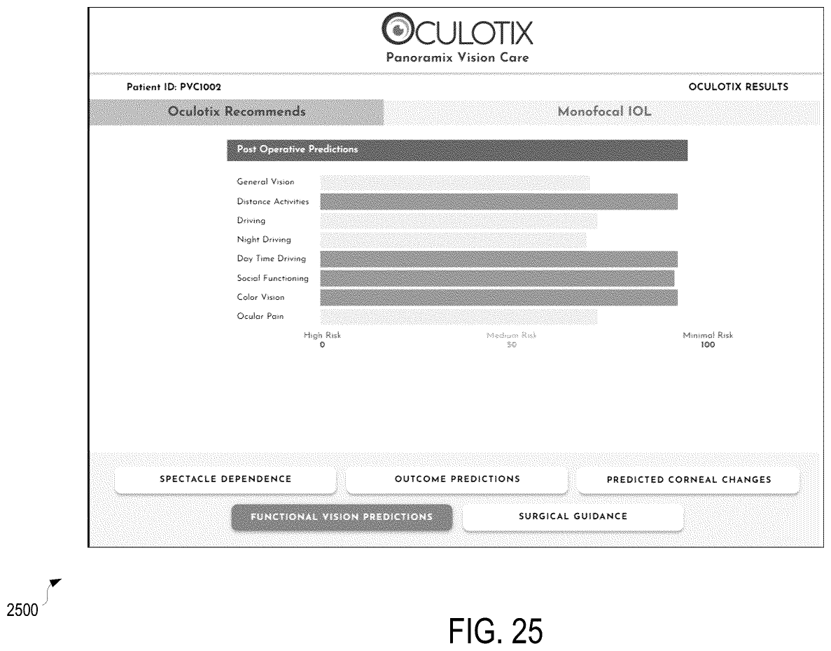

FIGS. 22-26 describe user interfaces presenting treatment recommendations and surgical guidance generated by a deep learning AI model as hosted via an ophthalmologic treatment platform as described herein. FIG. 22 illustrates a guidance user interface 2200 presenting surgery risks on a caregiver computing system, such as caregiver computing system 102. The surgery risks may include risks of compromise as to distance vision, intermediate vision, and near vision, as well as prediction accuracy for each. Additionally, as seen in the guidance user interface 2300 of FIG. 23, a recommendation or prediction regarding spherical power, cylindrical power, and cylinder axis may be presented. Still further, as seen in the guidance user interface 2400 of FIG. 24, a predicted endothelial count, a change in the endothelial count percentage, and corneal thickness may be depicted as well. Yet further, a prediction user interface 2500 of FIG. 25 may depict overall postoperative predictions regarding general vision, distance activities, driving, night driving, daytime driving, social functioning, color vision, and ocular pain may be presented. Each of these may be presented on a scale of minimal risk to high risk, as well as assigned a numerical score.

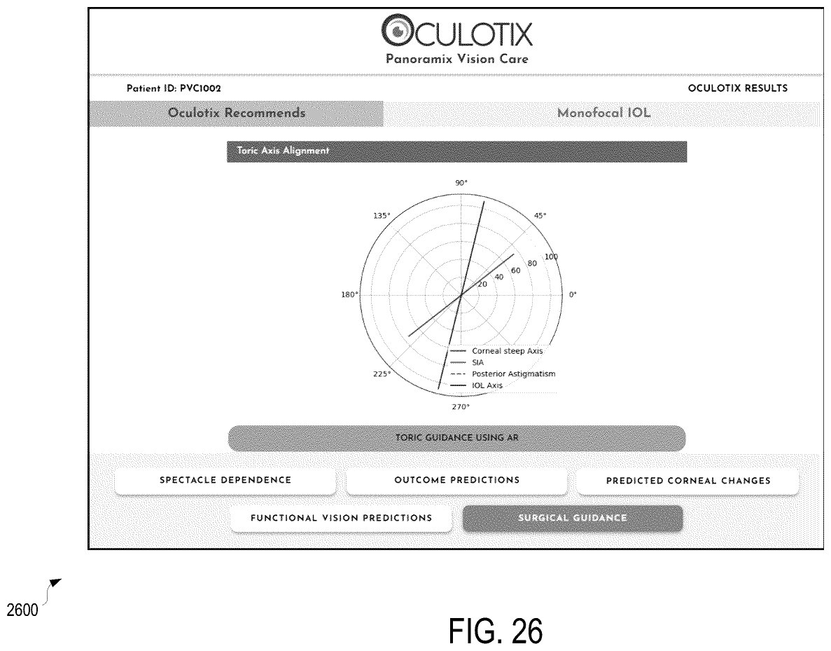

In addition to the predictions and recommendations, the user interfaces may further include a surgical guidance user interface 2600 as depicted in FIG. 26. As illustrated, the surgical guidance user interface 2600 may present optical characteristics for the patient, including the corneal steep axis, posterior astigmatism, and various other axes formed guidance of the surgeon. Additionally, an option may be presented to illustrate torque guidance using augmented reality, thereby assisting the physician during surgery itself. Various other guidance tools may be presented for operative guidance to the surgeon.

It is noted that other user interfaces may be provided to either the patient or to the caregiver depending on implementation. For example, other risk factors, alternative lenses, alternative operative procedures, and associated risks and potential outcomes illustrated alongside confidence levels in each.

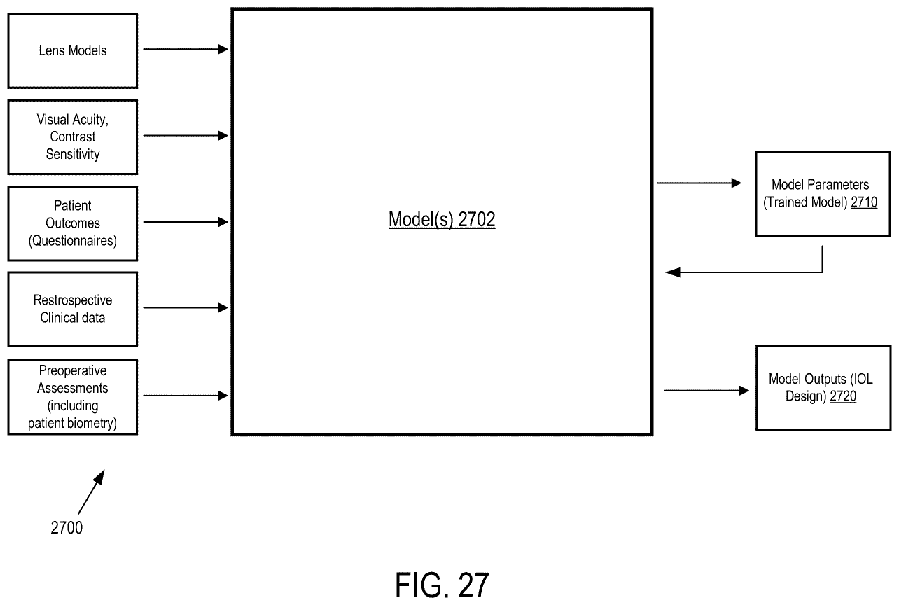

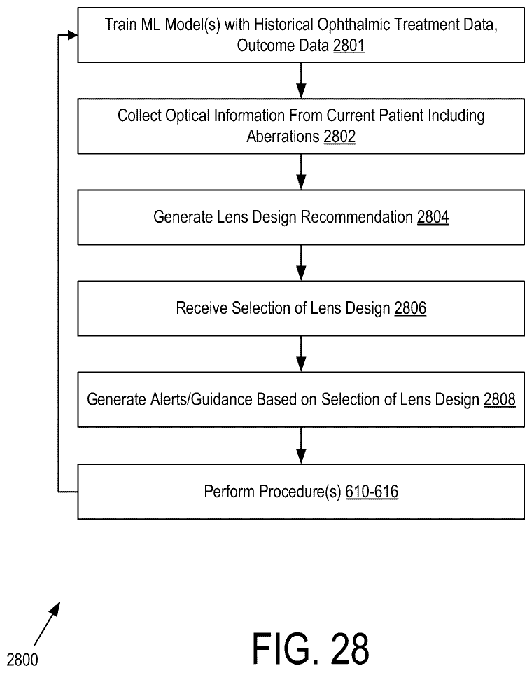

Referring now to FIGS. 27-28, further methods may be employed within the ophthalmologic treatment platform as described herein. In particular, a method and underlying modeling platform is provided which recommends lens models to patients based on lifestyle and post operative expectations. A surgeon or counselor is able to try to match the patient's profile with the best available match, in accordance with the above technologies. However, as an alternative or addition to the above, the ophthalmologic treatment platform is trained and configured to generate an optical design of an IOL that is customized to consider patient's lifestyle and post operative expectations along with the complete biometry of that patient. This allows the platform to be used to design a custom lens by taking into consideration the patient's corneal characteristics, such as aberration pattern of the patient's cornea, so as to design an intraocular lens which can optically deliver the outcomes such as extension in depth of focus.

In the example shown in FIG. 27, a modeling system 2700 that may be hosted on the ophthalmologic treatment platform may include one or more models 2702, which may be classifier models, such as neural networks and the like, which are useable to learn from retrospective post operative clinical data. For example, one or more existing lens models, as well as patient outcomes, retrospective clinical data, preoperative assessments for past users, and visual tests of past patients may be assessed. This may generate a set of model parameters 2710, which may be fed back to the model for purposes of generating predictions and/or classifications for a current patient. Preoperative assessments for the current patient may then be provided to trained models 2702, which can account for a variety of patient biometry or other parameters of the patient, and generates an optical design 2720 with specific sphere, cylinder, spherical aberration over the patient specific pupil size thus providing the patient with the outcomes which is intended by a specific lens design resulting in optimal visual outcomes for such a patient.

In example embodiments, the output of models 2702, i.e., the optical design 2720, may include a prediction of optimal lens parameters. In an example where models 2702 include one or more neural networks, such neural networks are trained on past patient biometry data and lens designs/outcomes to predict an optimal set of lens specifications (power, material, etc.) for a new patient based on their biometry and desired visual outcomes. This allows customization for each patient. Additionally, by analyzing past successful and unsuccessful lens designs for different patient biometrics, deep learning algorithms iteratively learn general strategies about how to design lenses tailored for certain types of patients. This knowledge can be used to guide new custom designs as well.

In some example implementations, in addition to generation of a proposed lens design, an ophthalmologic treatment platform that includes one or more trained models 2702 may generate suggested modifications to a selected design based on, for example, correlations and patterns in past patient data to help improve visual outcomes for patients with similar biometric profiles. Such correlations or patterns may not be apparent to the ophthalmologist or other user of a platform, for example due to volume of data or latent nature of the correlation.

Still further, in some instances the trained models 2702 may generate notifications regarding anomalies or outliner cases that may require particular design considerations. This may be due to particular rare combinations of biometric parameters. Such alerts may also not be apparent to the lens designer otherwise, and allow for extra customization and/or extra attention paid to unusual patient profiles.

Still further, in some instances, the trained models 2702 may estimate expected visual outcomes as noted above. For example, optical aberrations, glare or halo effects, and the like for a proposed custom lens design might be predicted, or at least a probability of the same might be provided. Such a prediction may be based on clinical outcomes of past patients with similar parameters, allowing the patient and technician to select an optimal design.

Still further, various other information may be used by the trained models 2702 to obtain lens designs that are optimal for a particular patient. For example, subjective data, such as lifestyle (L), personality type (P), Biometry (B), Ocular health (O), General health (H), aberration map (W) post operative vision expectations (E) are mapped into objective data as a function, e.g., f(L, P, B, O, H, W, E)×D (m), where D(m) is the learning coefficients from deep learning generated as training data 2710, and “x” represents a tensor product of those coefficients with the function defining the subjective data. The output is expected modulation transfer function values at different spatial frequencies to be able to meet the patient's expectations. The optical design at this point is constrained by wavefront aberrations of the cornea. The aberration map complimentary to the corneal aberration map centered around the visual axis is mapped onto the optical design to be custom generated. The initial optical design is redesigned within the pupil size of that patient so that the design intent of the lens such as extended depth of focus lens or bifocal or trifocal is customized for that patient.

The overall goal is to leverage the patterns within large datasets of past patient biometry and lens design/outcome information to provide data-driven insights and recommendations that help optimize custom lens design while accounting for each patient's specific visual goals and biometrics.

Referring to FIG. 28, a method 2800 for performing a custom lens design is provided that may be performed using the models 2702 described above, within the context of an ophthalmologic treatment platform, are described. The method 2800 may be performed using the systems and models described above in FIGS. 1-4, in example embodiments.

In the example as shown, the method 2800 includes training one or more machine learning models (step 2801). Training such models may include training deep learning models, neural networks, and/or other classifier models, with available treatment and outcome data from one or more past patients. The method 2800 further includes collecting optical information, such as patient optical geometry, a corneal aberration map, and various other patient information (step 2802). The other patient information may include a desired outcome, patient preferences, and other subjective factors as noted above, such as general health, lifestyle, personality type, and the like.

In the example shown, the method 2800 includes generating a lens design recommendation (step 2804). The lens design recommendation can be a new, customized lens design that is specific for the patient. The customize lens design may be derived from a model, such as models 2702, and presented to the patient and a technician for review. For example, the presentation of the optical lens design may be provided alongside other existing optical lens designs for purposes of comparison within a user interface. Example user interfaces are described above. Once the patient and technician determine a lens design to be used, a selection may be received at the platform (step 2806). Based on the selected lens design, one or more alerts or guidance may be generated by the platform (step 2808). Such alerts or recommendations may be specific to the patient, such as specific to the patient's lifestyle or biometry, specific to a particular procedure to be performed and used with the selected lens. Once selected, the particularized design may be communicated to a lens manufacturer (not shown) for creation prior to a surgical procedure. The lens design may be refractive or diffractive, and may be monofocal, bifocal, or have an extended depth of focus arrangement. An example of such an extended depth IOL design is described in U.S. Provisional Patent Application No. 63/616,352, filed on Dec. 29, 2023, the disclosure of which is hereby incorporated by reference in its entirety.

Continuing method 2800, additional method steps may be performed including performing a procedure to introduce the selected lens, perform postoperative monitoring on the patient, and provide feedback to the models to provide iterative, continuous learning and improvement regarding generation of recommendations as well as creation of new lens designs. Such steps 610-616 may be as described above in conjunction with FIG. 6, but as applied to a new/customized lens design.

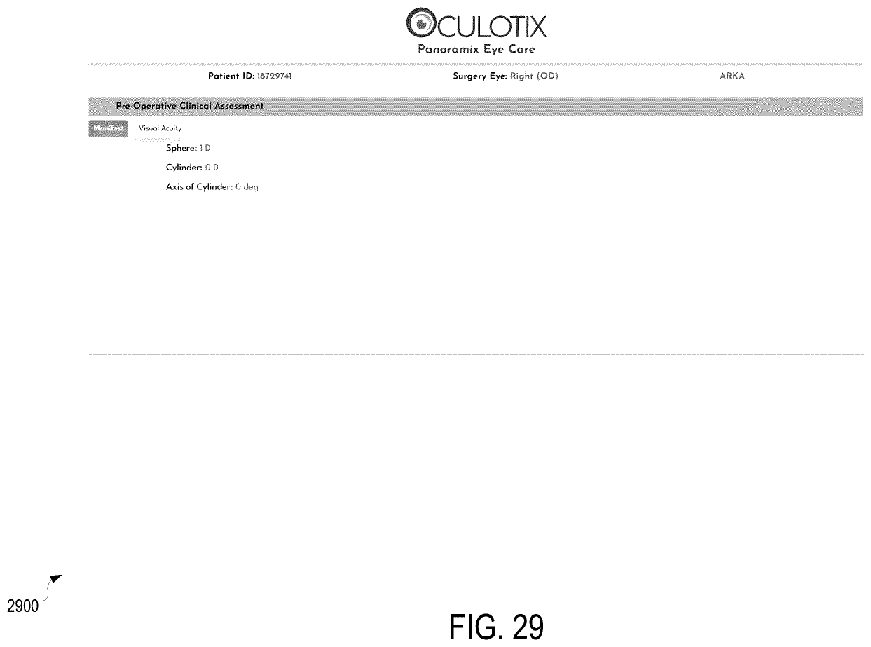

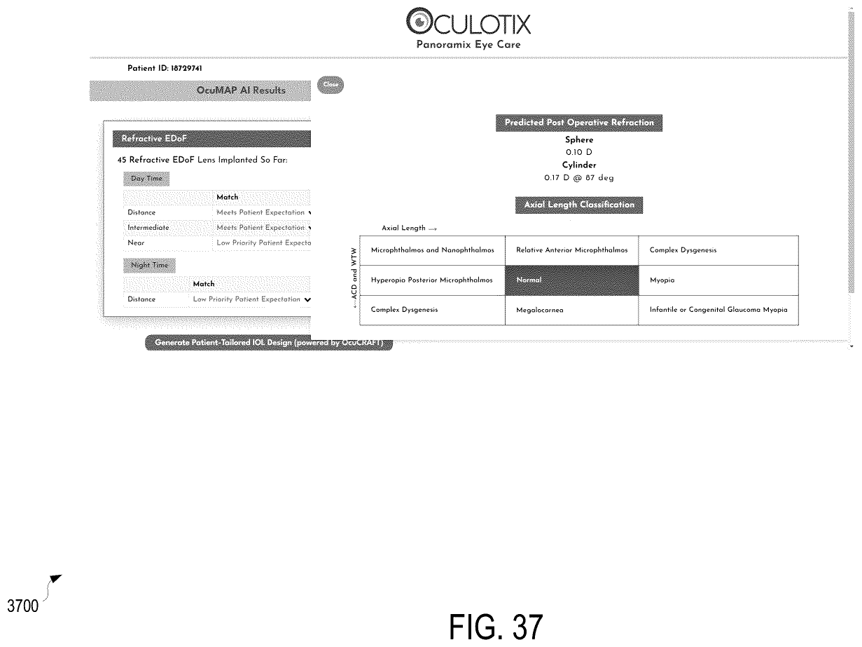

Referring to FIGS. 29-37, additional user interfaces are presented that may be generated in conjunction with usage of an ophthalmic treatment recommendation and guidance platform as described herein. In particular, the user interfaces represent alternative interfaces to those described above, and which may be used in conjunction with artificial intelligence-aided prediction of use of an IOL, and/or generation of recommended IOL designs optimized for patient outcomes.

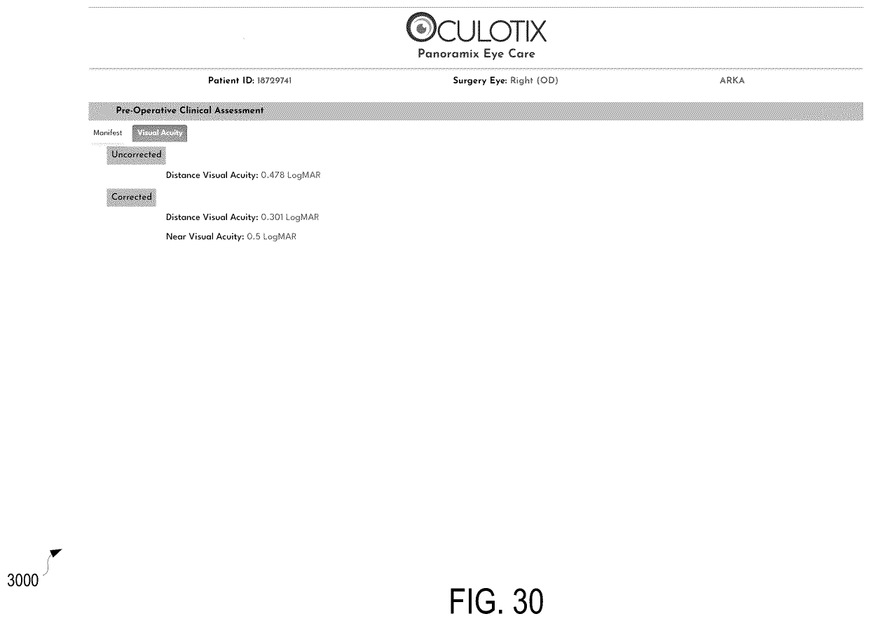

FIG. 29 illustrates a pre-operative manifest assessment user interface 2900 presented on the caregiver computing system based on interaction with an application hosted by the ophthalmic treatment recommendation and guidance platform, according to such a further example embodiment. As described above, the user interface 2900 may present pre-operative clinical assessment information for a particular patient, such as manifest information (sphere, cylinder, and axis information). Similarly, FIG. 30 illustrates a pre-operative visual acuity assessment user interface 3000 presented on the caregiver computing system based on interaction with an application hosted by the ophthalmic treatment recommendation and guidance platform. The user interface 3000 presents visual acuity information associated with the patient, including uncorrected and corrected distance and near visual acuity data.

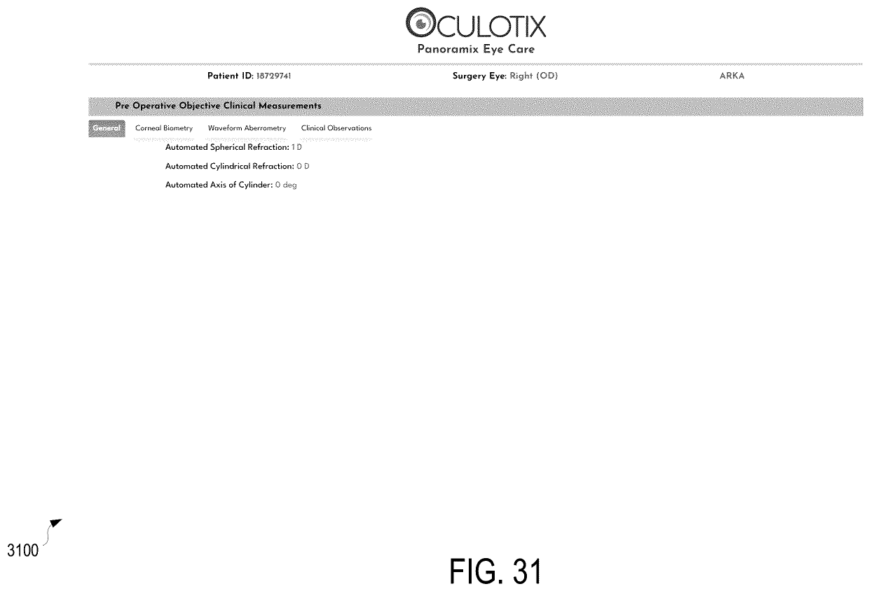

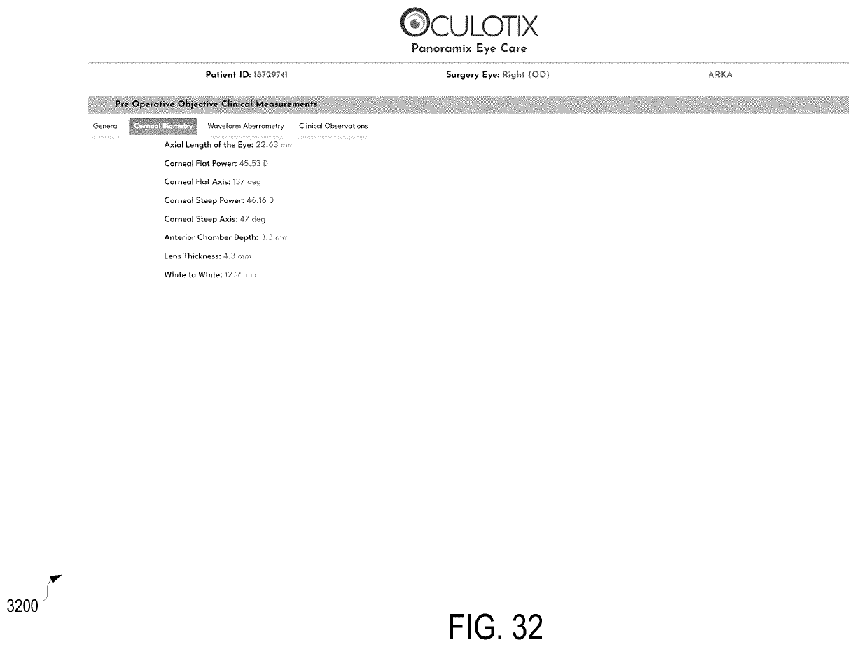

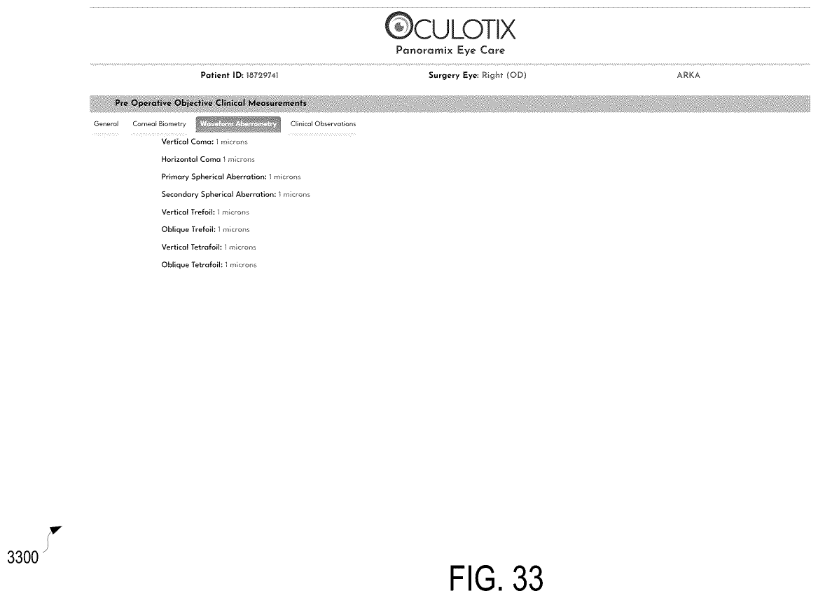

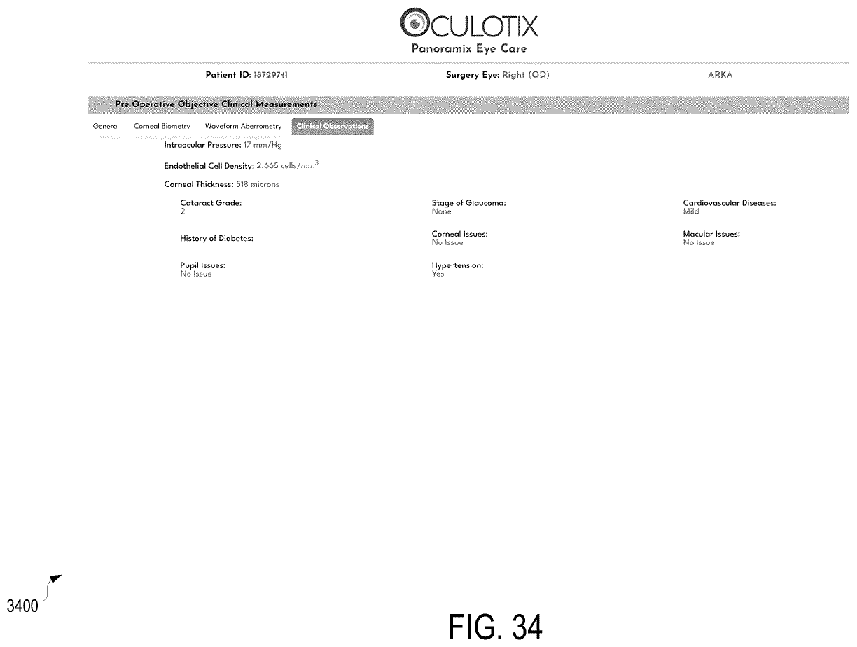

FIG. 31 illustrates a pre-operative clinical measurement user interface 3100 presented on the caregiver computing system based on interaction with an application hosted by the ophthalmic treatment recommendation and guidance platform, according to a further example embodiment. In this example, the user interface 3100 presents general preoperative objective clinical measurements, including automated spherical refraction, automated cylindrical refraction, and automated axis of cylinder measurements. Similarly, FIG. 32 illustrates a further pre-operative clinical measurement user interface 3200, which may depict corneal biometry data of the patient. Such corneal biometry data may include an axial length of the eye, a corneal flat power, a corneal flat axis, a corneal steep power, a corneal steep axis, and anterior chamber depth, a lens thickness, and a white to white distance. FIG. 33 illustrates a further pre-operative clinical measurement user interface 3300 that presents additional data of the patient, including, in particular, waveform aberrometry data. The waveform aberrometry data may include, for example, a vertical coma, a horizontal coma distance, a primary spherical aberration distance, a secondary spherical aberration distance, a vertical trefoil distance, an oblique tetrafoil distance, a vertical tetrafoil distance, and an oblique tetrafoil distance. Furthermore, FIG. 34 illustrates a further pre-operative clinical measurement user interface 3400 that presents various additional clinical observations that may be relevant to selection of an optimal IOL design. Such additional clinical observations may include an intraocular pressure, an endothelial cell density, a corneal thickness, as well as a cataract grade, stage of glaucoma, presence of cardiovascular diseases, history of diabetes, other corneal or macular issues, people issues, and the presence or absence of hypertension.

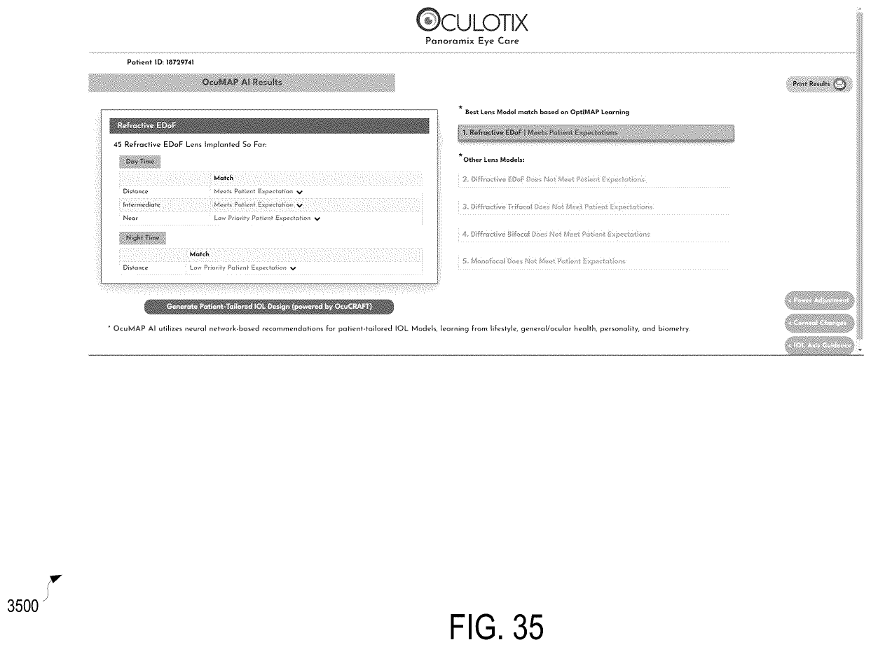

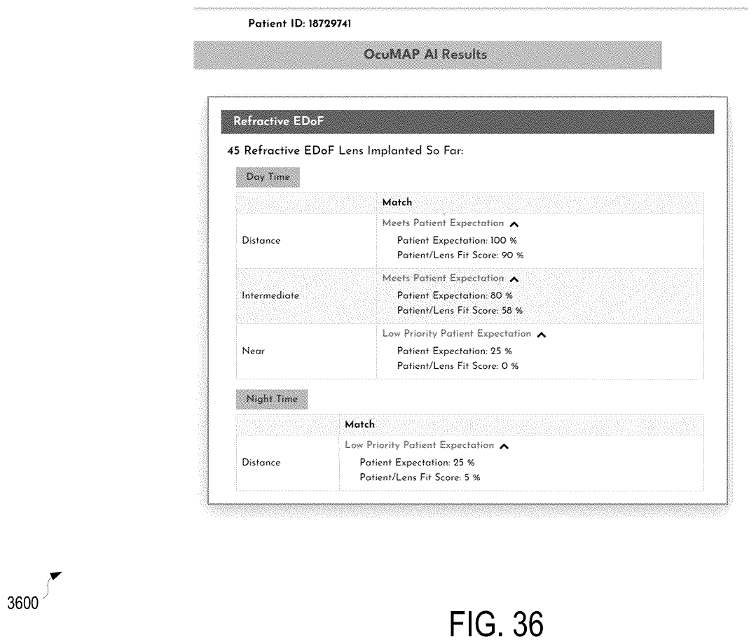

FIG. 35 illustrates a recommendation user interface 3500 presented on the caregiver computing system based on interaction with an AI-assisted lens matching and/or design application hosted by the ophthalmic treatment recommendation and guidance platform, according to such a further example embodiment. The recommendation user interface may represent the output of an artificial intelligence system, such as may operate on the platform as described above. In the example shown, a best lens model match may be generated for a particular patient, and preferred outcome predictions displayed, with respect to daytime and nighttime distance, intermediate, and near vision. Additionally, a patient tailored IOL design may be selected to be generated using the recommendation user interface 3500. In response to selection of such an option on the user interface, a neural network based artificial intelligence system may generate a particularized IOL design for the patient that optimizes patient outcomes, but is not constrained by the collection of currently available IOL designs, and instead is customized for that particular patient's biometry. FIG. 36 illustrates a detail user interface 3600 presented on the caregiver computing system in association with the recommendation illustrated in FIG. 35, according to that example embodiment. The detail user interface 3600 allows for detailed display of information relating to predicted outcome in response to use of a particular IOL design. Additionally, in response to selection of such results, a further pop up user interface 3700 representing a prediction user interface may display information including, but not limited to predicted post operative refraction.

While particular uses of the technology have been illustrated and discussed above, the disclosed technology can be used with a variety of data structures and processes in accordance with many examples of the technology. The above discussion is not meant to suggest that the disclosed technology is only suitable for implementation with the data structures shown and described above. For examples, while certain technologies described herein were primarily described in the context of queueing structures, technologies disclosed herein are applicable to data structures generally.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., operations, memory arrangements, etc.) described with respect to the figures herein are not intended to limit the technology to the particular aspects described. Accordingly, additional configurations can be used to practice the technology herein and/or some aspects described can be excluded without departing from the methods and systems disclosed herein.

Similarly, where operations of a process are disclosed, those operations are described for purposes of illustrating the present technology and are not intended to limit the disclosure to a particular sequence of operations. For example, the operations can be performed in differing order, two or more operations can be performed concurrently, additional operations can be performed, and disclosed operations can be excluded without departing from the present disclosure. Further, each operation can be accomplished via one or more sub-operations. The disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A computer-implemented method of managing treatment of an ophthalmic patient, the method comprising:
collecting optical information from a patient wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients; and
generating, at a first stage of treatment, a treatment recommendation regarding an ophthalmological treatment, the treatment recommendation including an ophthalmic lens type recommendation and a probability of a predetermined surgical outcome associated with the ophthalmic lens type recommendation.

2. The method of claim 1, wherein generating the treatment recommendation includes:
assigning a weight to each of the input parameters;
calculating a probability of a predetermined surgical outcome associated with each ophthalmic lens type of a plurality of different ophthalmic lens types;
generating the ophthalmic lens type recommendation for the patient; and
creating a mapping model of the recommended ophthalmic lens type.

3. The method of claim 1, further comprising:
generating, at the first stage of treatment, a plurality of probabilities of the predetermined surgical outcome associated with each of a plurality of different ophthalmic lens types; and
receiving a selection of the ophthalmic lens type from a user.

4. The method of claim 1, further comprising:
receiving optical procedure information at the ophthalmologic treatment platform, the optical procedure information being associated with an optical procedure selected for the patient; and
generating, at the deep-learning model, one or more optimizations for use during the ophthalmic procedure, the one or more optimizations being presented to a caregiver via an application exposed by the ophthalmic treatment platform.

5. The method of claim 4, further comprising generating one or more mechanical characteristics used during the ophthalmic procedure to mitigate risk of posterior capsular rupture.

6. The method of claim 4, further comprising:
receiving post-operative clinical assessment information regarding the patient at the ophthalmic treatment platform; and
generating, via the deep-learning model, one or more primary care predictions representing a predicted outcome of the ophthalmic procedure based, at least in part, on the post-operative clinical assessment information.

7. The method of claim 1, wherein the deep-learning model comprises a deep learning model including a neural network having a plurality of layers.

8. The method of claim 7, further comprising re-training the deep-learning model based on updated training data, the updated training data including ophthalmic procedure data associated with the patient, complication data associated with the patient, and patient survey data regarding treatment satisfaction of the patient.

9. The method of claim 1, wherein the ophthalmic treatment platform is configured to calculate one or more optical characteristics including lens spherical power, toric power, and aberrations;
wherein the deep-learning model is configured to generate the one or more primary care predictions by automatically compensating for changes in lens positions without the dependence on any manufacturer provided intraocular calculator constants.

10. The method of claim 1, wherein the ophthalmic procedure includes at least one of a cataract surgery, a retina surgery, a glaucoma surgery, a corneal transplant, and a LASIK surgery.

11. The method of claim 1, wherein the treatment recommendation includes a lens parameter adjustment.

12. The method of claim 1, wherein the historical ophthalmic procedure data includes pre-operative decisions and lens parameter adjustments for ophthalmic lenses selected for historical ophthalmic procedures, and wherein the deep-learning model is configured to generate a classification probability associated with each of a plurality of lens parameter adjustments such that:
$${y}_{v} = {k}_{0} + {\sum }_{i = 1}^{n} {x}_{i} {k}_{i} ; {y}_{v} = - 1 for {y}_{v} < 0 and {y}_{v} = + 1 for {y}_{v} > 0 ,$$
wherein “v” is a given input variable for a lens parameter, and in case of continuous variables where the values of yᵥ are non-binary, and wherein kᵢ represents a correlating weight that is proportional to the probability that the outcome predicted branch lies within the classes −1 or +1 or an analogous value x.

13. The method of claim 1, wherein the deep-learning model is adaptable in at least near realtime, and is fine tuned by computing rate of change of weights for each variable and assigning weighting for probability of each variable based on distances
$$\frac{\vert {y}_{v} - {C}_{- 1} \vert}{\vert {C}_{- 1} - {C}_{+ 1} \vert} and \frac{\vert {y}_{v} - {C}_{+ 1} \vert}{\vert {C}_{- 1} - {C}_{+ 1} \vert}$$
and probability multipliers scaled based on a preset threshold.

14. The method of claim 1, wherein the deep-learning model includes a plurality of classifiers and includes an output that is based, at least in part, on dependencies of current and previous information associated with a patient such that P(Oₙ)=Πᵢ₌₁ⁿP (Oᵢ|Oᵢ₋₁) where (Oₙ) is the outcome for the current patient based on the outcome Oᵢ₋₁ of patient i−1.

15. The method of claim 14, wherein the deep-learning model is trained to generate a probability of each of a set of outcomes O={o₁, o₂, o₃ . . . ot} where oᵢε{good outcome, bad outcome}, wherein each observation of the patient comes from an unknown state and will have an unknown sequence Q={q₁, q₂, q₃ . . . qₜ} where qiε{monofocal, extended depth of focus, bifocal, trifocal, quadrifocal, or any lens models or variations within any model to account for toricity or aberration profile}, to calculate
$$P ( {q}_{1} , {q}_{2} , {q}_{3} … {q}_{t} \vert {o}_{1} , {o}_{2} , {o}_{3} … {o}_{t} ) = \frac{P ( {o}_{1} , {o}_{2} , {o}_{3} … {o}_{t} \vert {q}_{1} , {q}_{2} , {q}_{3} … {q}_{t} ) P ( {q}_{1} , {q}_{2} , {q}_{3} … {q}_{t} )}{P ( {o}_{1} , {o}_{2} , {o}_{3} … {o}_{t} )}$$
corresponding to a combined probability used for decisionmaking by the deep-learning model for patient k such that
$${P}_{k =} \frac{\vert {y}_{v} - {C}_{n} \vert}{\vert {C}_{n} - {C}_{m} \vert} = - 1$$
for Pk<0 and +1 for Pₖ>0 and is not used in the decision-making process if Pₖ=0.

16. The method of claim 15, wherein each classifier is selectively excluded based on whether a confidence level is below a predetermined threshold.

17. The method of claim 1, wherein collecting optical information from a patient includes applying at least one of an optical measurement device or an ultrasound device to a low dense cataract patient, and wherein the deep-learning model is used to generate a predicted corrected axial length.

18. The method of claim 1, wherein collecting optical information from a patient includes determining an estimated difference in vitreoretinal interface in a high dense cataract patient.

19. An ophthalmic treatment recommendation and guidance platform implemented on a computing system comprising:
a processor;
a memory communicatively coupled to the processor, the memory storing instructions that, when executed by the processor, cause the platform to:
collect optical information from a patient wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients; and
generate, at a first stage of treatment, a treatment recommendation regarding an ophthalmological treatment, the treatment recommendation including an ophthalmic lens type recommendation and a probability of a predetermined surgical outcome associated with the ophthalmic lens type recommendation.

20. The ophthalmic treatment recommendation and guidance platform of claim 19, further comprising an application exposed to a treatment provider, the application presenting a user interface including at least the ophthalmic lens type recommendation and one or more potential surgical outcomes.

21. A computer-implemented method of managing treatment of an ophthalmic patient, the method comprising:
collecting optical information from a patient, wherein the optical information includes one or more input parameters including eye dimensional measurements, the optical information being received as input parameters by an ophthalmic treatment platform hosting a deep-learning model, the deep-learning model being trained using training data including historical ophthalmic procedure data associated with a plurality of patients, complication data associated with the plurality of patients, and patient survey data regarding treatment satisfaction of the plurality of patients; and
generating, at the deep-learning model, a treatment recommendation regarding an ophthalmological treatment, the treatment recommendation including a custom intraocular lens design recommendation.

22. The computer-implemented method of claim 21, wherein the custom intraocular lens design recommendation corresponds to at least one of a refractive lens design or a diffractive lens design.

23. The computer-implemented method of claim 21, further comprising, in response to selection of the custom intraocular lens design recommendation, generation of one or more alerts or recommendations regarding treatment to optimize a patient outcome.
